(19) United States
(12) Patent Application Publication
(10) Pub. No.: US 2023/0405364 A1
(43) Pub. Date: Dec. 21, 2023

(54) ULTRASOUND ABLATION APPARATUS AND METHODS OF USE

(71) Applicant: Current Surgical, Inc., Washington, DC (US)

(72) Inventors: Alireza MASHAL, Washington, DC (US); Christopher Robert WAGNER, Washington, DC (US)

(21) Appl. No.: 18/099,240

(22) Filed: Jan. 19, 2023

Related U.S. Application Data
(63) Continuation of application No. PCT/US2021/042248, filed on Jul. 19, 2021.
(60) Provisional application No. 63/053,898, filed on Jul. 20, 2020.

(51) Int. Cl. A61N 7/02; A61B 8/12; B06B 1/02

(52) U.S. Cl. CPC A61N 7/022; A61N 2007/0039; B06B 1/0292; A61B 8/12

(57) ABSTRACT

An ultrasound ablation apparatus is disclosed for facilitating navigation, ablation, and ablation monitoring relative to an at least one target material positioned within or adjacent to a tissue of a patient. In at least one embodiment, the apparatus provides an instrument portion and a base portion engaged with a proximal end of the instrument portion. An opposing distal end of the instrument portion provides a plurality of ultrasound transducers configured for both obtaining an at least one ultrasound image of the target material and selectively emitting acoustic energy to heat, destroy and/or perturb the target material, the transducers arranged so as to form an at least one array. The distal end of the instrument portion further provides an at least one acoustically matched covering positioned and configured for extending over top of the transducers so as to not inhibit the functionality of the transducers.

### CROSS-REFERENCE

This application is a continuation of PCT Application No. PCT/US2021/042248, filed Jul. 19, 2021; which claims priority to Provisional Application No. 63/053,898, filed Jul. 20, 2020; which are incorporated herein by reference in their entirety and to which application we claim priority under 35 USC § 120.

### BACKGROUND

The subject of this patent application relates generally to ablation devices, and more particularly to an ultrasound ablation apparatus and associated methods of use for facilitating navigation, ablation, and ablation monitoring.

Applicant hereby incorporates herein by reference any and all patents and published patent applications cited or referred to in this application.

By way of background, ablation is a minimally invasive surgical technique where a surgical probe (such as a needle, for example) is inserted into or near offending tissue—such as a cancerous lesion, damaged nerve, or nerve(s) with abnormal neuronal activity, malfunctioning cardiac tissue, disruptive growths such as uterine fibroids, etc. (hereinafter referred to generally as “target material” for simplicity purposes)—and energy is delivered through the tip of the probe, which destroys the target material. A number of technologies have been used to deliver such energy at the tip of the probe, including RF energy, microwave energy, steam, and cold. However, existing ablation techniques suffer from several limitations that limit the surgeon's ability to (1) confirm the location of the ablation tool in relation to the target material, and (2) control and confirm the extent of energy delivery throughout the target material. These limitations cause surgeons to make a number of tradeoffs when it comes to delivering therapy. For example, in cancer therapy, the inability to precisely control the treatment zone leads to a risk of not completely treating the target material. Thus surgeons often choose to over-deliver energy to ensure the entire extent of the target material is destroyed. Similarly, over-delivery of energy can be used to overcome lack of precision in positioning the ablation tool directly in the center of the target material. This has the obvious drawback of destroying healthy tissue and limiting the utility of such techniques.

One solution to this lack of precision has been to use external imaging, both to image the location of the ablation tool with respect to the target material, as well as identify the extent of energy delivery in real time. An example of such a technique is MRI thermometry, where an ablation procedure is performed within the bore of an MM, and the MRI can detect temperature changes within tissue due to a range of temperature sensitive magnetic resonance parameters (e.g., T1 and T2 relaxation times and proton resonance frequency). However, MRI guided procedures suffer from two shortcomings: an increase in procedure complexity due to the need for MM compatible surgical tools, and the limited availability of MRIs. Other external 3D imaging modalities with similar resolutions, such as CT scanning, also cause related procedural burdens (i.e., difficulty of carrying out surgery in a CT scanner, exposure of patient and clinical staff to harmful radiation, and limited availability of equipment).

Another external imaging approach to aid both localization and temperature monitoring has been to use ultrasound imaging. Ultrasound is a viable temperature monitoring modality since acoustic properties of tissue—such as speed of sound and attenuation—are temperature dependent. In addition to these intrinsic temperature dependent properties, as tissue is ablated, its stiffness also changes, which can be detected by ultrasound. A number of different strategies have been investigated to monitor ablation procedures by exploiting these various contrast mechanisms; however, they are fundamentally limited in several ways. First, ultrasound imaging loses resolution when imaging deeper into tissue. Second, ultrasound requires a continuous region of tissue with similar stiffness between the transducer and the structure being imaged (an “acoustic window”); thus, ultrasound-based ablation monitoring is limited to applications in superficial anatomical structures (e.g., breast). Third, the temperature dependent tissue properties being exploited for ultrasound thermometry are subtle (e.g., <˜5% change for speed of sound over clinically relevant temperature changes). Combined with the non-linearities and diffraction present in ultrasound propagation, the temperature accuracy achieved with traditional ultrasound thermometry has limited clinical utility.

Furthermore, the use of a separate, non-integrated monitoring system, such as those described above, still presents significant challenges. For example, it requires the clinicians to react to observed temperature changes and adjust treatment as needed (e.g., change power settings, physically move surgical probes, etc.). This type of reactive nature further limits the benefits of the pairing of external imaging and current ablation devices.

Ablation devices can only be used as a first line treatment option if they can simultaneously satisfy 3 functions: (1) local tool-tip placement with respect to the target material, (2) precise ablation shaping, and (3) real-time ablation treatment monitoring. Specifically, the latter two features must be integrated in a closed-loop manner to achieve the desired benefit. The current state of the art has focused on combining various applicators with external imaging, such as CT, Mill, and traditional ultrasound. However, the known prior art has failed to successfully combine the above three functions into a single integrated surgical device.

Existing needle-based ablations deliver energy locally, with the needle central in the region of delivered energy. This has several limitations. First, when placing the needle into a tumor, this can cause an increase in the tumor interstitial pressure, increasing the likelihood of tumor rupture. Second, placing the needle within the tumor can cause tumor cells to attach to the needle, leading to needle track seeding (where tumor cells are distributed along the path of needle insertion, outside of the tumor body), thus limiting the ability of the clinician to reuse the needle during the same operation at a new location. This is one of the reasons multiple needles are required per operation, increasing costs of the operation.

All current local solid tumor treatments, including ablation, rely on identifying the physical anatomic location of the tumor within the body. Ideally, this identification is sensitive, specific, spatially accurate, can be carried out without harm to the patient, and can be carried out simultaneously with therapy delivery to accurately guide the therapy. Unfortunately, however, current diagnostic tools each provide only a fraction of these features. Clinicians therefore combine multiple approaches, which increases complexity, inaccuracy, and likelihood of error. For example, CT scans provide a high resolution 3D image of a person's anatomy, and certain cancers appear as variations in intensity within that image. However, the intensity variation of the tumor with respect to the surrounding tissue may be indistinct. While contrast enhanced imaging can improve the intensity difference, the image still represents a single moment in time. Thus, the spatial configuration of the tumor and the surrounding soft tissue changes when delivering therapy (especially surgical manipulations). Also, the intensity variation observed does not correlate exactly with the extent of the cellular boundary of the tumor, nor are the intensity variations highly sensitive and specific to metastatic (as opposed to benign) growths. Finally, a CT scan exposes patients to significant doses of radiation. Similarly, tissue biopsies provide a different set of tradeoffs for characterizing and locating tumors. They are the gold standard for sensitivity and specificity, and for certain therapies there is an opportunity to take a biopsy at the time of therapy delivery. However, arriving at a characterization decision from a tissue sample is slow (from 20-60 minutes), which limits utility when guiding therapy decisions mid-therapy. Further, the biopsy is at a single spatial location, so is poorly suited to characterizing the spatial distribution of metastatic cells. Finally, lab based diagnostics can be very sensitive and specific to certain pathological indicators, but again require a slow process of drawing blood, then sending to a dedicated lab facility for preparation and analysis.

One critical gap in these diagnostic and characterization methods is the in-situ ability to assess spatial distributions of tissue structure in relation to other anatomy (e.g., proximity to other organs), that includes overall shape of pathological tissue distributions, and at a cellular level of resolution. Histological analysis of biopsy tissue achieves cellular level resolution and understanding of cellular morphology, but removed from a shape and location understanding of the anatomy. CT scans and other 3D imaging modalities such as MM give an understanding of larger scale anatomy and pathology location, but poor spatial resolution and tissue discrimination abilities. Another gap is the in-situ ability to understand functional relationships and responses. For all methods described above, each measurement is taken at a single moment in time. It is difficult to characterize tissue over time; nor can the tissue or pathology be characterized in response to a stimulus. Thus, there remains a need for an apparatus that is capable of combining cellular resolution evaluation similar to biopsies, while also understanding larger scale pathological tissue distributions and relationships with other anatomy, delivering this understanding over time, while in-situ, and concomitant with therapy delivery. If measured with the therapy delivery, and/or paired with outcome measurements over time, these measurements could also be used to predict efficacy of certain treatments. Furthermore, if such an apparatus could derive these measurements simultaneously with the existing state of the art (i.e., biopsies+3D imaging), that would provide a bridge of clinical evidence leading to adoption.

Aspects of the present invention fulfill these needs and provide further related advantages as described in the following summary.

It should be noted that the above background description includes information that may be useful in understanding aspects of the present invention. It is not an admission that any of the information provided herein is prior art or relevant to the presently claimed invention, or that any publication specifically or implicitly referenced is prior art.

### SUMMARY

Aspects of the present invention teach certain benefits in construction and use which give rise to the exemplary advantages described below.

The present invention solves the problems described above by providing an ultrasound ablation apparatus for facilitating navigation, ablation, and ablation monitoring relative to an at least one target material positioned within or adjacent to a tissue of a patient. In at least one embodiment, the apparatus provides an instrument portion and a base portion engaged with a proximal end of the instrument portion. An opposing distal end of the instrument portion provides a plurality of ultrasound transducers configured for both obtaining an at least one ultrasound image of the target material and selectively emitting acoustic energy to heat, destroy and/or perturb the target material, the transducers arranged so as to form an at least one array. The distal end of the instrument portion further provides an at least one acoustically matched covering positioned and configured for extending over top of the transducers so as to not inhibit the functionality of the transducers.

In at least one other embodiment, the apparatus provides an instrument portion and a base portion engaged with a proximal end of the instrument portion. An opposing distal end of the instrument portion provides a plurality of ultrasound transducers configured for both obtaining an at least one ultrasound image of the target material and selectively emitting acoustic energy to heat and destroy the target material, the transducers arranged so as to form an at least one array positioned on a sidewall of the instrument portion. The sidewall provides an at least one acoustically matched covering positioned and configured for extending over top of the transducers so as to not inhibit the functionality of the transducers while retaining a substantially circular cross-section for the distal end of the instrument portion. The at least one covering is in fluid communication with an at least one reservoir containing an acoustic medium configured for facilitating acoustic communication between the transducers and the target material.

In at least one other embodiment, the apparatus provides an instrument portion and a base portion engaged with a proximal end of the instrument portion. An opposing distal end of the instrument portion provides a plurality of ultrasound transducers configured for both obtaining an at least one ultrasound image of the target material and selectively emitting acoustic energy to heat and destroy the target material, the transducers arranged so as to form an at least one array positioned on a terminal face of the distal end of the instrument portion. The terminal face of the distal end of the instrument portion provides an at least one acoustically matched covering positioned and configured for extending over top of the transducers so as to not inhibit the functionality of the transducers. The at least one covering is in fluid communication with an at least one reservoir containing an acoustic medium configured for facilitating acoustic communication between the transducers and the target material.

In use, the apparatus is capable of precisely focusing acoustic energy toward the target material to achieve a desired ablation shape, based on data gathered from the at least one ultrasound image.

Other features and advantages of aspects of the present invention will become apparent from the following more detailed description, taken in conjunction with the accompanying drawings, which illustrate, by way of example, the principles of aspects of the invention.

### BRIEF DESCRIPTION OF THE DRAWINGS

The accompanying drawings illustrate aspects of the present invention. In such drawings:

FIG. 1 is a simplified schematic view of an exemplary ultrasound ablation apparatus in communication with each of an exemplary computing device and an exemplary imaging display, in accordance with at least one embodiment;

FIG. 2 is a diagrammatic view of an instrument portion of an exemplary ultrasound ablation apparatus, in accordance with at least one embodiment;

FIG. 3 is a diagrammatic view of an instrument portion of a further exemplary ultrasound ablation apparatus, in accordance with at least one embodiment;

FIG. 4 is a diagrammatic view of an instrument portion of a still further exemplary ultrasound ablation apparatus, in accordance with at least one embodiment;

FIG. 4A is a cross-sectional view taken along line 4A-4A of FIG. 4;

FIG. 5 is a diagrammatic view of an instrument portion of a still further exemplary ultrasound ablation apparatus, in accordance with at least one embodiment;

FIGS. 6A-6C are diagrammatic views of an instrument portion of a still further exemplary ultrasound ablation apparatus, in accordance with at least one embodiment;

FIG. 7 is a diagrammatic view of a still further exemplary ultrasound ablation apparatus, in accordance with at least one embodiment;

FIG. 7A is a cross-sectional view taken along line 7A-7A of FIG. 7;

FIG. 8 is a diagrammatic view of an instrument portion of a still further exemplary ultrasound ablation apparatus, in accordance with at least one embodiment;

FIG. 9 is a diagrammatic view of an instrument portion of a still further exemplary ultrasound ablation apparatus, in accordance with at least one embodiment;

FIG. 10 is a diagrammatic view of an instrument portion of a still further exemplary ultrasound ablation apparatus, in accordance with at least one embodiment;

FIG. 11 is a cross-sectional view of an instrument portion of a still further exemplary ultrasound ablation apparatus, in accordance with at least one embodiment;

FIG. 12 is a perspective view of an instrument portion of a still further exemplary ultrasound ablation apparatus, in accordance with at least one embodiment;

FIG. 12A is a cross-sectional view taken along line 12A-12A of FIG. 12;

FIG. 13 is a diagrammatic view of an instrument portion of a still further exemplary ultrasound ablation apparatus, in accordance with at least one embodiment;

FIG. 13A is a cross-sectional view taken along line 13A-13A of FIG. 13;

FIG. 14 is a diagrammatic view of an instrument portion of a still further exemplary ultrasound ablation apparatus, in accordance with at least one embodiment;

FIG. 15 is a diagrammatic view of an instrument portion of a still further exemplary ultrasound ablation apparatus, in accordance with at least one embodiment;

FIG. 16 is a diagrammatic view of an instrument portion of a still further exemplary ultrasound ablation apparatus, in accordance with at least one embodiment;

FIG. 17 is a diagrammatic view of a still further exemplary ultrasound ablation apparatus inserted into a patient, in accordance with at least one embodiment;

FIG. 18 is a diagrammatic view of a still further exemplary ultrasound ablation apparatus positioned within a vessel of a patient, in accordance with at least one embodiment;

FIG. 19 is a diagrammatic view of a still further exemplary ultrasound ablation apparatus positioned within an organ of a patient, in accordance with at least one embodiment;

FIG. 20 is a diagrammatic view of a still further exemplary ultrasound ablation apparatus, in accordance with at least one embodiment;

FIGS. 21 and 22 are electrical schematic views of a still further exemplary ultrasound ablation apparatus, in accordance with at least one embodiment;

FIG. 23 is a diagrammatic view of an instrument portion of a still further exemplary ultrasound ablation apparatus, in accordance with at least one embodiment;

FIG. 24 is a diagrammatic view of a plurality of transducers of an exemplary ultrasound ablation apparatus, in accordance with at least one embodiment;

FIG. 25 is a diagrammatic view of an instrument portion of a still further exemplary ultrasound ablation apparatus, in accordance with at least one embodiment;

FIGS. 26 and 27 are diagrammatic views of an instrument portion of a still further exemplary ultrasound ablation apparatus, in accordance with at least one embodiment;

FIGS. 28 and 29 are diagrammatic views of an instrument portion of a still further exemplary ultrasound ablation apparatus, in accordance with at least one embodiment;

FIG. 30 is a diagrammatic view of an instrument portion of a still further exemplary ultrasound ablation apparatus, in accordance with at least one embodiment;

FIG. 31 is a diagrammatic view of an instrument portion of a still further exemplary ultrasound ablation apparatus, in accordance with at least one embodiment; and

FIG. 32 is a diagrammatic view of an instrument portion of an exemplary ultrasound ablation apparatus being removed from a wound of a patient, in accordance with at least one embodiment.

The above described drawing figures illustrate aspects of the invention in at least one of its exemplary embodiments, which are further defined in detail in the following description. Features, elements, and aspects of the invention that are referenced by the same numerals in different figures represent the same, equivalent, or similar features, elements, or aspects, in accordance with one or more embodiments.

### DETAILED DESCRIPTION

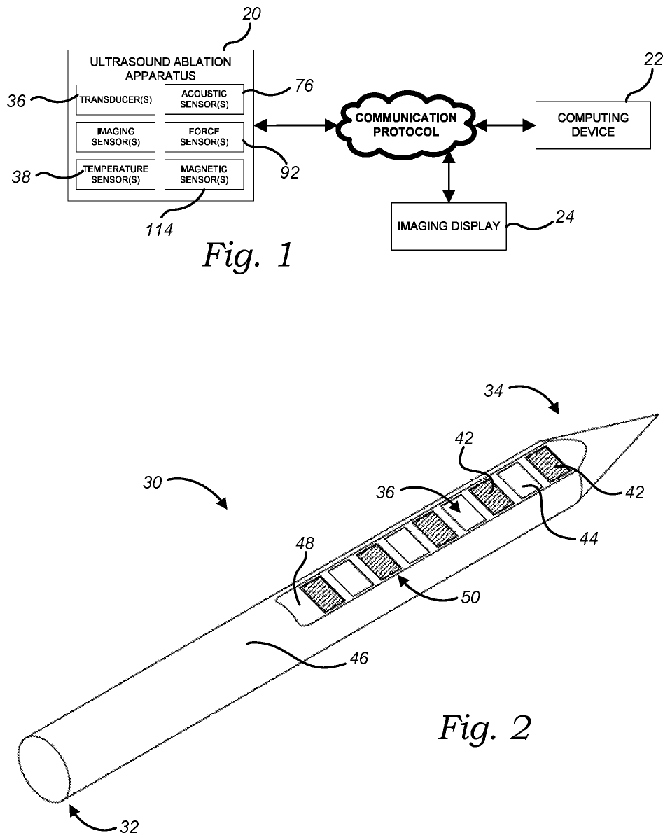

Turning now to FIG. 1, there is shown a simplified schematic view of an exemplary ultrasound ablation apparatus 20 in selective communication with each of an exemplary computing device 22 and an exemplary imaging display 24, in accordance with at least one embodiment. At the outset, it should be noted that while the apparatus 20 is depicted predominantly as a needle in many of the accompanying drawings for illustrative purposes, in further embodiments, the apparatus 20 may be configured as any other type of hand tool, now known or later developed, for virtually any context where the need for such an ultrasound ablation apparatus 20 exists. Thus, the present invention should in no way be limited to the specific configurations of the apparatus 20 shown and described herein.

In at least one embodiment, as discussed in greater detail below, the apparatus 20 incorporates components to facilitate navigation, ablation, and ablation monitoring relative to an at least one target material 26. In that regard, it should be noted that while certain embodiments of the apparatus 20 are illustrated in the accompanying drawings for illustrative purposes, in further embodiments, the apparatus 20 may take on any other size, shape or dimensions, or may be constructed out of any material (or combination of materials), now known or later developed.

In at least one embodiment, the apparatus 20 is in selective communication with at least one of an at least one computing device 22 and an at least one imaging display 24, the at least one computing device 22 and imaging display 24 configured for receiving and displaying the ultrasound images transmitted by the apparatus 20. In at least one embodiment, the at least one computing device 22 is further configured for storing the ultrasound images transmitted by the apparatus 20. It should be noted that communication between each of the apparatus 20, at least one computing device 22, and at least one imaging display 24 may be achieved using any wired- or wireless-based communication protocol (or combination of protocols) now known or later developed. As such, the present invention should not be read as being limited to any one particular type of communication protocol, even though certain exemplary protocols may be mentioned herein for illustrative purposes. It should also be noted that the term “computing device” is intended to include any type of computing or electronic device now known or later developed having a display screen (or at least in communication with a display screen), such as desktop computers, mobile phones, smartphones, laptop computers, tablet computers, personal data assistants, gaming devices, wearable devices, etc. As such, the present invention should not be read as being limited to use with any one particular type of computing device, even though certain exemplary devices may be mentioned or shown herein for illustrative purposes. Additionally, the term “imaging display” is intended to include any type of standalone display device now known or later developed, such as a television, a display screen, heads-up-display enabled glasses or goggles, etc. As such, the present invention should not be read as being limited to use with any one particular type of imaging display, even though certain exemplary devices may be mentioned or shown herein for illustrative purposes.

In at least one embodiment, the apparatus 20 provides a base portion 28 and an instrument portion 30 engaged with the base portion 28. In at least one embodiment, a proximal end 32 of the instrument portion 30 is removably engaged with the base portion 28. In at least one alternate embodiment, the proximal end 32 of the instrument portion 30 is permanently engaged or otherwise integral with the base portion 28. In at least one embodiment, as illustrated in FIG. 2, an opposing distal end 34 of the instrument portion 30 provides an at least one ultrasound transducer 36 positioned and configured for both obtaining an at least one ultrasound image of the target material 26 (along with surrounding tissue 40) and selectively emitting acoustic energy to heat, destroy and/or perturb the target material 26. In at least one such embodiment, the at least one transducer 36 is configured for operating in a pulse-echo configuration—i.e., it is configured to emit and subsequently receive ultrasonic pulses in order to obtain the at least one ultrasound image. In at least one alternate embodiment, the distal end 34 of the instrument portion 30 provides an at least one first transducer 42 configured for obtaining an at least one ultrasound image of the target material 26 (along with surrounding tissue 40), along with an at least one second transducer 44 configured for selectively emitting acoustic energy to heat, destroy and/or perturb the target material 26. In at least one such alternate embodiment, the at least one first transducer 42 and at least one second transducer 44 are arranged in an alternating pattern within the distal end 34 of the instrument portion 30. In at least one embodiment, the at least one transducer 36 is a high-bandwidth, low-profile, high-efficiency transducer 36, such as a capacitive micromachined ultrasound transducer 36 (“CMUT”). In further embodiments, the at least one transducer 36 may be any other type of ultrasound device, now known or later developed, capable of allowing the apparatus 20 to substantially carry out the functionality described herein. As described further below, the quantity and arrangement of transducers 36 may vary between embodiments, thereby enabling the apparatus 20 to be configured for effectively accessing various types and locations of target material 26.

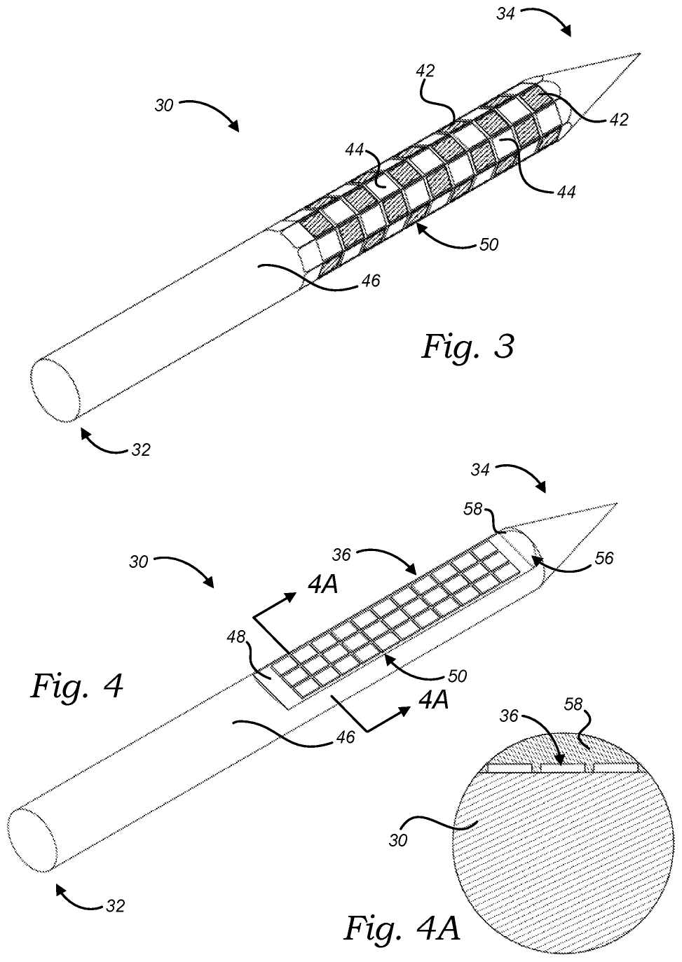

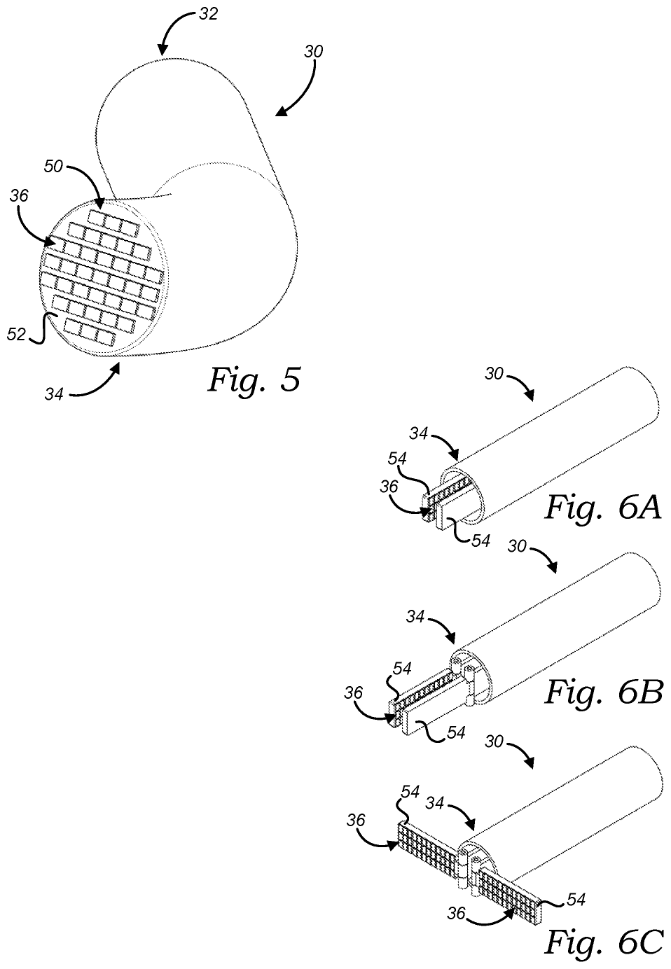

In at least one embodiment, the transducers 36 are positioned on a sidewall 46 of the distal end 34 of the instrument portion 30, so as to be arranged in a “side facing” configuration—i.e., where the sidewall 46 is to be positioned adjacent to the target material 26. In at least one such embodiment, as illustrated in FIG. 2, the transducers 36 are arranged in a one-dimensional array 50 along a length of the sidewall 46. In at least one alternate embodiment, as illustrated in FIG. 3, the transducers 36 are arranged in a two-dimensional array 50 along both the length and a circumference of the sidewall 46. In at least one further such embodiment, as illustrated in FIGS. 4 and 4A, the sidewall 46 provides a substantially flat cut-out 48 on which the transducers 36 are positioned, thereby allowing the transducers 36 to be arranged in a substantially flat array 50. In at least one further embodiment, as illustrated in FIG. 5, the transducers 36 are positioned on a terminal face 52 of the distal end 34 of the instrument portion 30, so as to be arranged in a “forward facing” configuration—i.e., where the terminal face 52 is to be positioned adjacent to the target material 26. In at least one still further embodiment, as illustrated in FIGS. 6A-6C, the transducers 36 are positioned on an at least one articulating arm 54 provided by the distal end 34 of the instrument portion 30 and configured for selectively pivoting and/or rotating relative to the distal end 34 so as to direct the transducers 36 toward the target material 26. Thus, for example, an array 50 of transducers 36 that has a planar field of view can be rotated to achieve a full view around the distal end 34 of the instrument portion 30. In at least one such embodiment, the array 50 of transducers 36 can be swept back and forth through a subset of angles (i.e., less than 360 degrees) for a more constrained field of view. Control of these articulations can be coordinated with the imaging or energy delivery functions of the transducers 36, to achieve a larger field of view. In at least one such embodiment, the articulating arm 54 is further configured for moving between a retracted position (FIG. 6A)—wherein the articulating arm 54 is positioned substantially within the distal end 34 of the instrument portion 30—and a deployed position (FIG. 6C)—wherein the articulating arm 54 extends a distance out from the distal end 34 of the instrument portion 30. In that regard, it should be noted that the sizes, shapes, dimensions, configurations, relative positions, and quantities of the at least one transducer 36 as depicted in the drawings (and as described herein) are merely exemplary. In further embodiments, each of the at least one transducer 36 may take on any other sizes, shapes, dimensions, configurations, relative positions, and/or quantities, now known or later developed, so long as the apparatus 20 is capable of substantially carrying out the functionality described herein. Similarly, in further embodiments, each of the at least one articulating arm 54 may take on any other sizes, shapes, dimensions, configurations, relative positions, and/or quantities, now known or later developed, so long as the apparatus 20 is capable of substantially carrying out the functionality described herein.

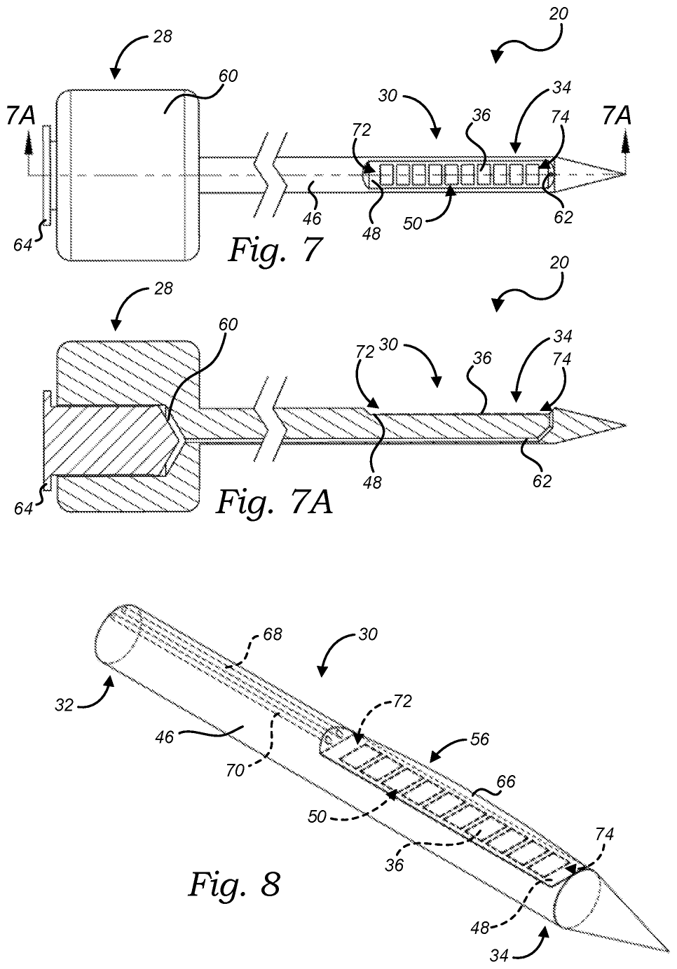

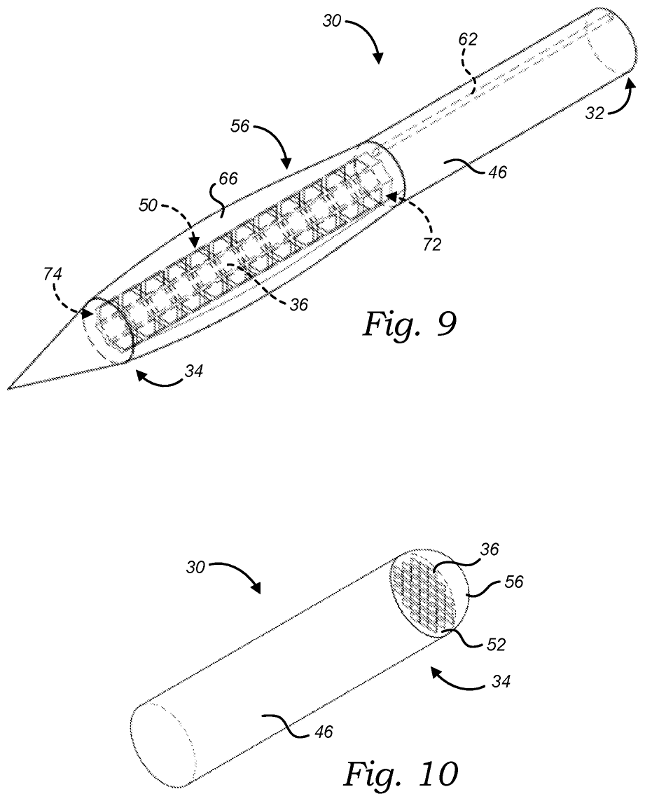

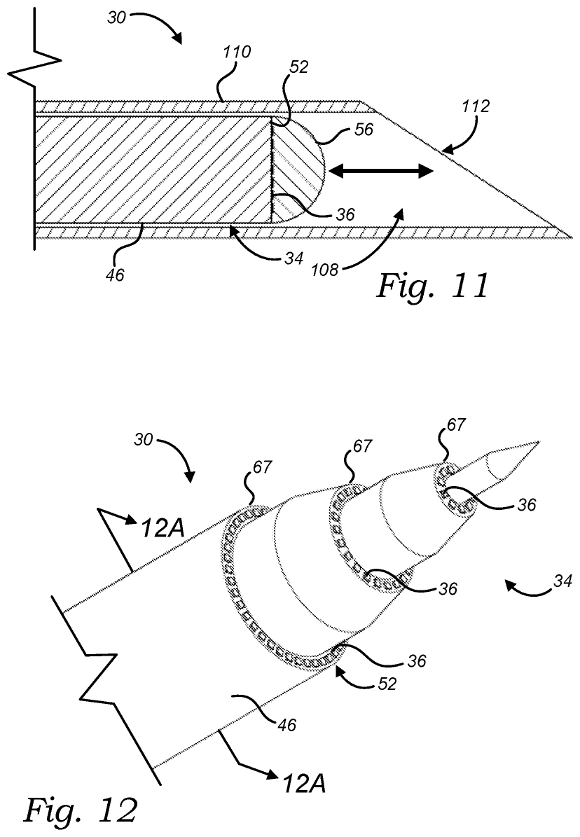

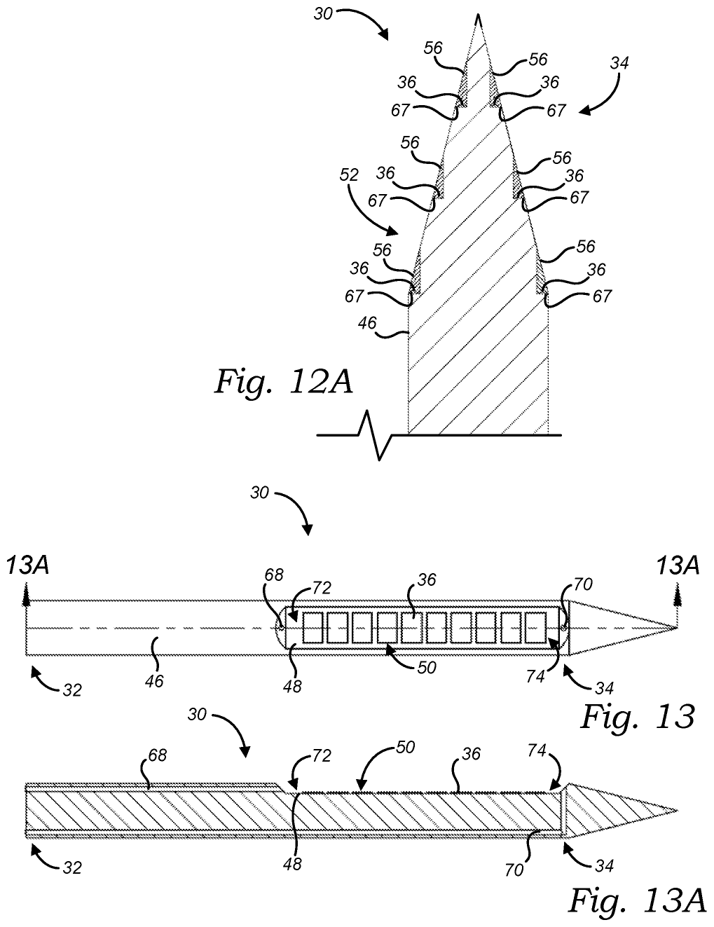

Maintenance of acoustic contact between the at least one transducer 36 and the target material 26 is critical to efficient functioning of the apparatus 20. Accordingly, in at least one embodiment, the sidewall 46 provides an at least one acoustically matched covering 56 positioned and configured for extending over top of the transducers 36 so as to not inhibit the functionality of the transducers 36 while retaining a substantially circular cross-section for the distal end 34 of the instrument portion 30. In at least one such embodiment, as illustrated in FIGS. 4 and 4A, where the sidewall 46 provides a cut-out 48, the at least one covering 56 is a rigid shield 58 configured for being selectively retractable when the transducers 36 are positioned proximal the target material 26, thereby temporarily exposing the transducers 36 to the bodily fluids that surround the target material 26, such that the bodily fluids facilitate acoustic communication between the transducers 36 and the target material 26. In at least one alternate embodiment, the distal end 34 of the instrument portion 30 is configured for selectively delivering an acoustic medium upon the shield 58 being retracted, such that the acoustic medium facilitates acoustic communication between the transducers 36 and the target material 26. In at least one such embodiment, the acoustic medium is at least one of a fluid (such as water or saline, for example) or a gel (such as ultrasound gel, for example). In at least one such embodiment, as illustrated in FIGS. 7 and 7A, the acoustic medium is contained in an at least one reservoir 60 positioned within the instrument portion 30 or the base portion 28 of the apparatus 20, with the at least one reservoir 60 being in fluid communication with the distal end 34 of the instrument portion 30 via an at least one delivery channel 62 extending therebetween. In at least one alternate embodiment, the reservoir 60 is positioned external to the apparatus 20. In at least one embodiment, the acoustic medium is selectively delivered from the reservoir 60 to the distal end 34 of the instrument portion 30—either manually or automatically via a plunger 64, pump or other mechanism now known or later developed. It should be noted that the sizes, shapes, dimensions, configurations, relative positions, and quantities of the at least one reservoir 60 as depicted in the drawings (and as described herein) are merely exemplary. In further embodiments, each of the at least one reservoir 60 may take on any other sizes, shapes, dimensions, configurations, relative positions, and/or quantities, now known or later developed, so long as the apparatus 20 is capable of substantially carrying out the functionality described herein. In at least one further embodiment, as illustrated in FIG. 8, the at least one covering 56 is a deformable enclosure 66 positioned and configured for covering the transducers 36 so as to maintain the acoustic medium over top of the transducers 36 while retaining a substantially circular cross-section for the distal end 34 of the instrument portion 30. In at least one such embodiment, where the instrument portion 30 provides a cut-out 48 on which the transducers 36 are positioned, the enclosure 66 extends over top of the cut-out 48 so as to retain a substantially circular cross-section for the distal end 34 of the instrument portion 30. In at least one alternate such embodiment, as illustrated in FIG. 9, where the transducers 36 are arranged in a two-dimensional array 50 along both the length and circumference of the sidewall 46, the enclosure 66 extends circumferentially around the distal end 34 of the instrument portion 30, again, so as to retain a substantially circular cross-section for the distal end 34 of the instrument portion 30. In at least one further alternate embodiment, as illustrated in FIG. 10, where the transducers 36 are positioned on the terminal face 52 of the distal end 34 of the instrument portion 30, so as to be arranged in a “forward facing” configuration, the covering 56 is shaped as a substantially hemispherical dome and positioned to extend over top of the terminal face 52. In at least one further such embodiment, as illustrated in FIG. 11, the distal end 34 of the instrument portion 30 is positioned within an internal passage 108 of a needle 110. In such embodiments, the distal end 34 of the instrument portion 30 is configured for selectively extending a distance toward a terminal opening of the internal passage 108 of the needle 110 in order to observe, heat, destroy and/or perturb the target material 26 when the needle 110 is inserted into the tissue 40, then subsequently retracting back into the internal passage 108 prior to the needle 110 being withdrawn from the tissue 40. In at least one still further alternate embodiment, as illustrated in FIGS. 12 and 12A, where the transducers 36 are positioned on the terminal face 52 of the distal end 34 of the instrument portion 30, the terminal face 52 of the distal end 34 is configured as a sharp, stepped needle providing a plurality of circumferential, coaxially aligned steps 67 oriented in a “forward facing” direction, with each of the steps 67 having a diameter relatively smaller than a diameter of an immediately preceding one of the steps 67. In at least one such embodiment, the terminal face 52 of the distal end 34 further provides a plurality of transducers 36 radially arranged along each of the steps 67. Additionally, in at least one such embodiment, as illustrated in FIG. 12A, the covering 56 is positioned and configured for extending over top of each of the steps 67 and associated transducers 36 so as to cooperate with the distal end 34 to form a substantially conical shape for the distal end 34. In still further embodiments, the at least one steps 67 may take on any other sizes, shapes, dimensions, configurations, relative positions, and/or quantities, now known or later developed, so long as the apparatus 20 is capable of substantially carrying out the functionality described herein. Additionally, in still further embodiments, the at least one covering 56 may take on any other sizes, shapes, dimensions, configurations, relative positions, and/or quantities, now known or later developed, (dependent, at least in part, on the arrangement of transducers 36 on the instrument portion 30) so long as the apparatus 20 is capable of substantially carrying out the functionality described herein. In at least one still further embodiment, the enclosure 66 may be omitted altogether.

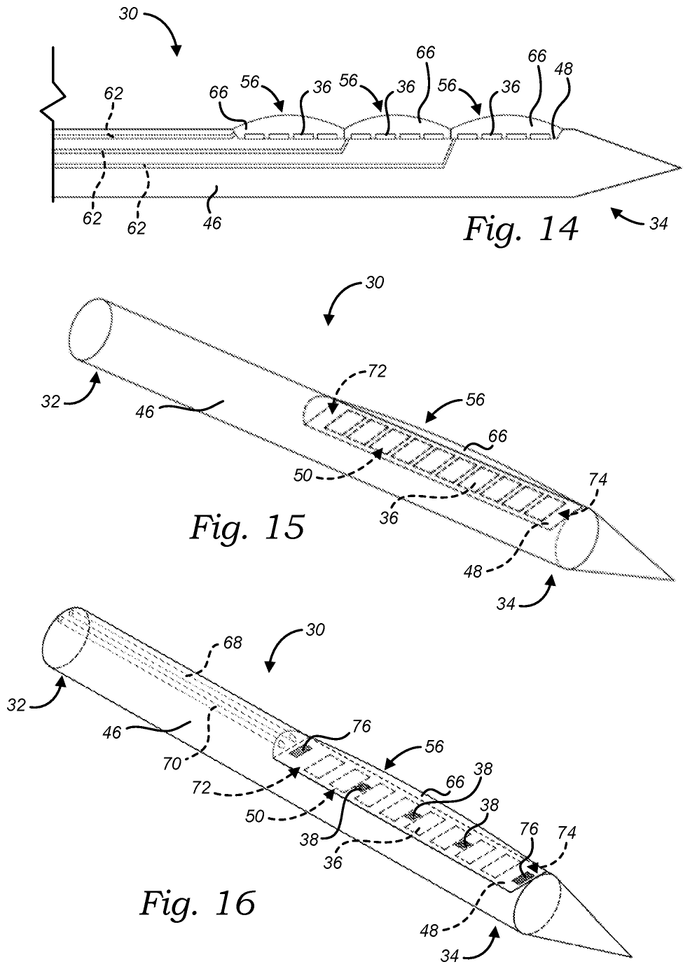

Referring again to FIG. 8, in at least one embodiment, the apparatus 20 provides a first delivery channel 68 and a second delivery channel 70 each extending between the at least one reservoir 60 and the distal end 34 of the instrument portion 30 so as to circulate the acoustic medium therethrough. In at least one such embodiment, as illustrated in FIG. 8, each of the first delivery channel 68 and second delivery channel 70 terminate proximal a first end 72 of the array 50 of transducers 36—or, alternatively, proximal an opposing second end 74 of the array 50 of transducers 36, such that the insertion action of the distal end 34 of the instrument portion 30 (in combination with the resistance provided by the tissue 40) draws the acoustic medium along the array 50 of transducers 36. In at least one alternate such embodiment, as illustrated in FIGS. 13 and 13A, the first delivery channel 68 terminates proximal the first end 72 of the array 50 of transducers 36 while the second delivery channel terminates proximal the second end 74 of the array 50 of transducers 36. In at least one further embodiment, as illustrated in FIG. 14, the sidewall 46 provides a plurality of coverings 56 each positioned and configured for extending over top of a subset of the transducers 36, with each of the coverings 56 being in fluid communication with the at least one reservoir 60 via a separate at least one delivery channel 62. In at least one such embodiment, by differentially regulating a pressure (as determined by an at least one pressure sensor in at least one such embodiment) the apparatus 20 is capable of optimizing acoustic contact for different tissue 40 shapes, as well as applying different pressure distributions to the tissue 40, then image the corresponding deformation. This allows for a technique known as elastography (discriminating tissue 40 based on its stiffness) and overcomes a traditional limitation of elastography where only a single deformation can be applied, from a single direction. In still further embodiments, the at least one delivery channel 62 may take on any other sizes, shapes, dimensions, configurations, relative positions, and/or quantities, now known or later developed, (dependent, at least in part, on the arrangement of transducers 36 on the instrument portion so long as the apparatus 20 is capable of substantially carrying out the functionality described herein. In at least one alternate embodiment, as illustrated in FIG. 15, the at least one delivery channel 62 is omitted, such that the at least one enclosure 66 is pre-filled with the acoustic medium. In an at least one further alternate embodiment, the at least one enclosure 66 is constructed out of a solid deformable acoustic medium (such as silicone, for example) rather than being fillable with a fluid- or gel-based acoustic medium.

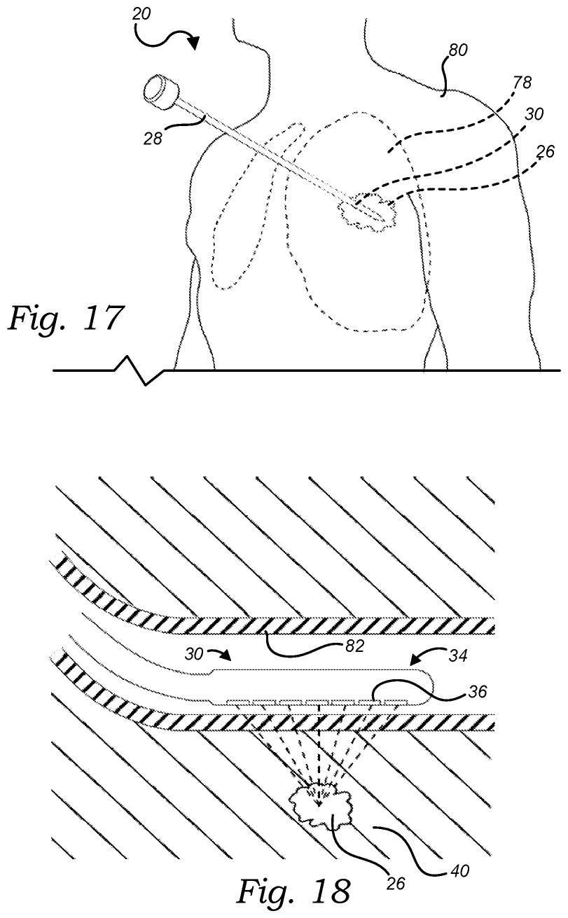

In at least one embodiment, as illustrated in FIG. 16, the distal end 34 of the instrument portion 30 further provides an at least one contact sensor 76 (e.g., pressure sensor, or optical reflectivity) positioned and configured for detecting the presence of acoustic contact between the at least one transducer 36 and the target material 26 through the covering 56. The at least one contact sensor 76 may also be configured for processing the at least one ultrasound image. In at least one such embodiment, the at least one contact sensor 76 is positioned within the covering 56. In further embodiments, the at least one contact sensor 76 may take on any other sizes, shapes, dimensions, configurations, relative positions, and/or quantities, now known or later developed, (dependent, at least in part, on the arrangement of transducers 36 on the instrument portion 30) so long as the apparatus 20 is capable of substantially carrying out the functionality described herein. Additionally, in at least one embodiment, the distal end 34 of the instrument portion 30 further provides an at least one temperature sensor 38 positioned and configured for detecting a temperature of the distal end 34 of the instrument portion 30 along with the tissue 40 proximal thereto. Accordingly, in at least such embodiment, upon the detected temperature reaching a pre-defined threshold, the apparatus 20 is capable of utilizing the acoustic medium to selectively cool the tissue 40 proximal to the distal end 34 of the instrument portion 30 by selectively circulating the acoustic medium through the distal end 34, as described above. In this way, the apparatus 20 is able to keep the tissue 40 proximal the instrument portion healthy while the target material 26 is ablated. In at least one such embodiment, the at least one temperature sensor 38 is positioned within the covering 56. In further embodiments, the at least one temperature sensor 38 may take on any other sizes, shapes, dimensions, configurations, relative positions, and/or quantities, now known or later developed, (dependent, at least in part, on the arrangement of transducers 36 on the instrument portion 30) so long as the apparatus 20 is capable of substantially carrying out the functionality described herein. Accordingly, in at least one such embodiment, the at least one transducer 36 is capable of both obtaining the at least one ultrasound image of the target material 26 (along with surrounding tissue 40) and selectively emitting acoustic energy to heat, destroy and/or perturb the target material 26, all while maintaining a safe distance from the target material 26 and any sensitive anatomy proximal thereto. As illustrated in the accompanying drawings and described further below, the apparatus 20 may embody a variety of configurations depending on the context in which the apparatus 20 is to be utilized—dependent at least in part of the type and location of the target material 26. In at least one embodiment, as illustrated in FIG. 17, the instrument portion 30 is configured as a needle, while the base portion 28 is configured as a manually operated handle, with the needle deployed percutaneous into an organ 78 of a patient 80 containing the target material 26. In at least one such embodiment, the needle has an outer diameter of between approximately 1 mm and approximately 4 mm; however, in further such embodiments, the needle may have any other outer diameter, dependent at least in part on the context in which the apparatus 20 is to be utilized. Additionally, in at least one such embodiment, the needle is constructed out of a rigid material, such as stainless steel for example; however, in further such embodiments, the needle may be constructed out of any other rigid or resilient material (or combinations of such materials) now known or later developed, dependent at least in part on the context in which the apparatus 20 is to be utilized.

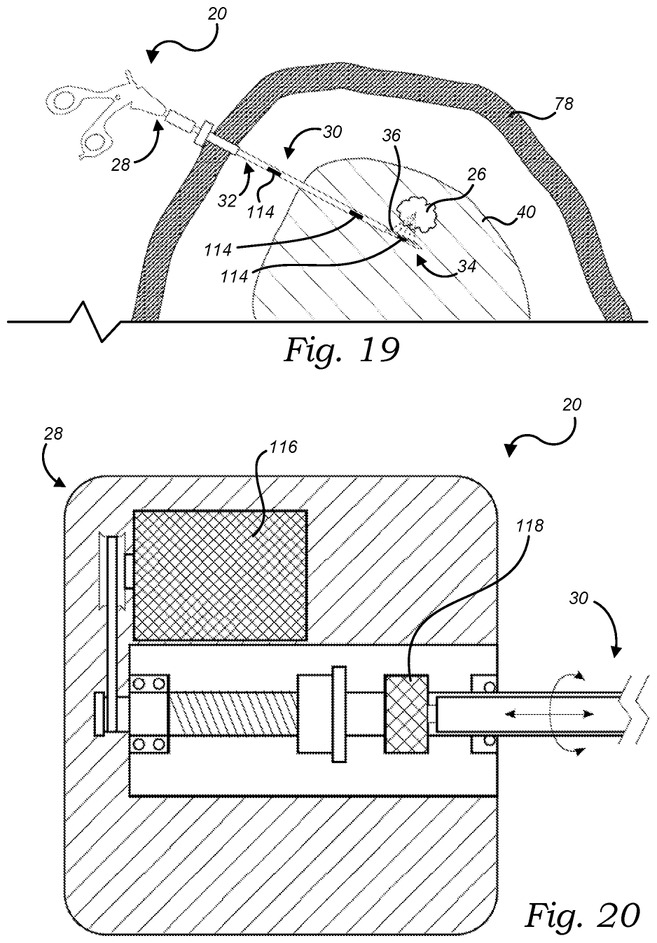

In at least one alternate embodiment, as illustrated in FIG. 18, the instrument portion 30 is configured for being inserted into and traversing through a catheter 82, endoscope or similar structure, allowing the distal end 34 of the instrument portion 30 to reach the target material 26 by utilizing minimally invasive or natural orifice access to internals of the body. In such embodiments, the instrument can deliver energy into a target material without damaging the tissue lumen (e.g., vessel wall or esophageal wall). In at least one further alternate embodiment, as illustrated in FIG. 19, the instrument portion 30 is configured as a laparoscopic surgical tool, for use in traditional minimally invasive surgical procedures. Similarly, the apparatus 20 can be configured as a robotic surgical tool for use with a surgical robot, with the distal end 34 of the instrument portion 30 located distal to potentially articulating joints. In such embodiments, the apparatus 20 is operated remotely and/or automatically via the surgical robot. In at least one still further alternate embodiment, the apparatus may be configured as a deployable mechanism through a working channel, as illustrated in FIGS. 6A-6C, where in the deployed configuration the at least one transducer 36 is able to contact a wider range of tissue 40, or achieve a more favorable angle with respect to the tissue 40. The deployment may be manual or robotically controlled. In at least one such robotic embodiment, the deployment of articulations of the apparatus 20 are also operated remotely and/or automatically via the surgical robot. In at least one embodiment, as illustrated in FIG. 20, the base portion 28 provides an insertion motor 116 in mechanical communication with the instrument portion 30 and configured for selectively driving the distal end 34 of the instrument portion 30 a distance into (and subsequently out of) the tissue 40 during use of the apparatus 20. Additionally, in at least one such embodiment, the base portion 28 provides a rotation motor 118 in mechanical communication with the instrument portion and configured for selectively rotating the distal end 34 of the instrument portion 30 in order to align the at least one transducer 36 with the target material 26. Thus, in such embodiments, the even a manually held apparatus 20 can acquire a full 360 degree volumetric image (at the distal end 34 of the instrument portion 30) without having to manually reorient the apparatus 20. It should be noted that the size, shape, dimensions and position of each of the insertion motor 116 and the rotation motor 118 relative to the base portion 28 as depicted in the drawings is merely exemplary, such that in further embodiments, each of the insertion motor 116 and the rotation motor 118 may take on any other size, shape and/or dimensions, now known or later developed, and may be positioned and/or arranged elsewhere on or within the base portion 28, so long as the apparatus 20 is capable of substantially carrying out the functionality described herein. In at least one still further embodiment, one or both of the insertion motor 116 and the rotation motor 118 may be omitted altogether in lieu of a mechanism configured for enabling manual interaction with the instrument portion 30 to achieve the same functionality. Accordingly, the term “motor” as used herein is intended to include any such manual mechanisms, now known or later developed. Additionally, the sizes, shapes, dimensions, quantities and positions of the gears mechanically linking each of the insertion motor 116 and the rotation motor 118 with the instrument portion 30, as depicted in the drawings, is merely exemplary, such that in further embodiments, the gears may take on any other sizes, shapes, dimensions and/or quantities, now known or later developed, and may be positioned and/or arranged elsewhere on or within the base portion 28, so long as the apparatus 20 is capable of substantially carrying out the functionality described herein. In each of these embodiments, a key benefit is that the instrument portion 30 can be inserted into tissue 40, such that ablations of the target material 26 can be carried out and monitored without the need for direct visualization (and the associated collateral tissue 40 damage). Further, when the distal end 34 of the instrument portion 30 is embedded within the tissue 40, acoustic contact is maintained even during small motions of the tissue 40. In still further embodiments, again, the apparatus 20 may be configured as any other type of hand tool, now known or later developed, for virtually any context where the need for such an ultrasound ablation apparatus 20 exists.

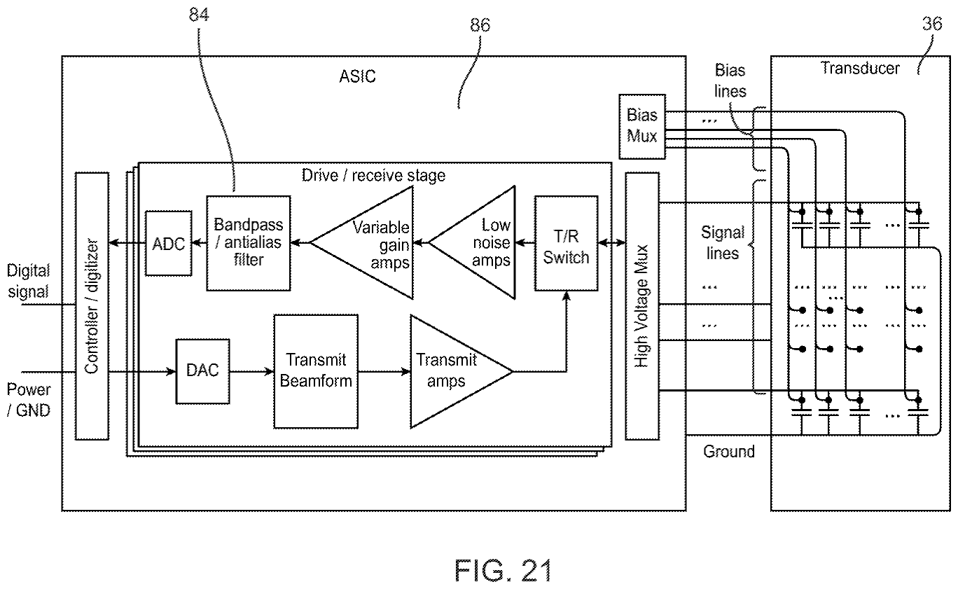

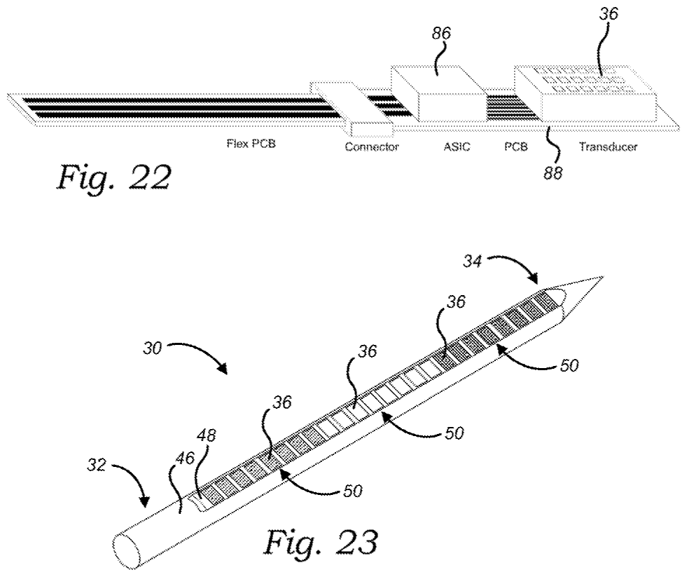

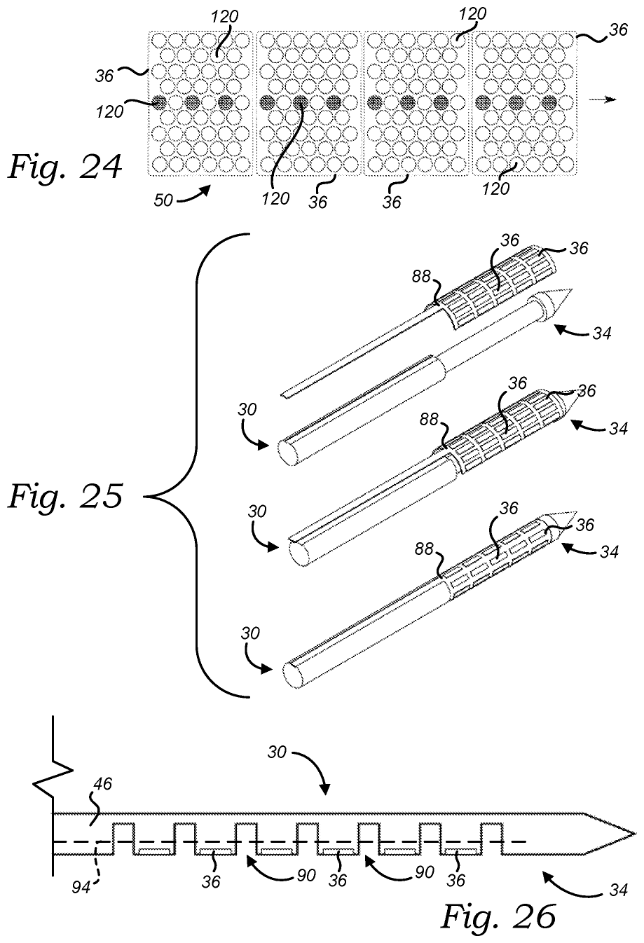

In at least one embodiment, the apparatus 20 utilizes several techniques that enable a small, compact configuration. In at least one embodiment, as illustrated in FIG. 21, the instrument portion 30 provides a plurality of conductors positioned and configured for electrically interconnecting electronic drivers 84 with the transducers 36. One key difficulty in controlling an array 50 of transducers 36 on the instrument portion 30 (particularly when the instrument portion 30 is configured as a needle) is the relatively small cross-sectional area of the instrument portion 30, limiting the number of conductors available to uniquely address transducers 36. In at least one embodiment, where each transducer 36 is a CMUT, the instrument portion 30 further provides a plurality of switches or relays into the CMUT fabrication, to allow for multiplexing the addressing of the transducers 36 (or the arrays 50 of transducers 36). In at least one such embodiment, one set of wires allows access to rows of an array 50 of transducers 36, while a further set of wires allows access to columns of said array 50 of transducers 36. In this manner, a single transducer 36 in an n×m array 50 of transducers 36 can be accessed with only n+m wires (as opposed to n*m in traditional single-wire-per-transducer 36 configurations). In at least one alternate embodiment, as illustrated in FIGS. 21 and 22, the instrument portion 30 provides traditional ASIC microchips 86 to provide some portion of the multiplexing or preprocessing of the transducer 36 drive signal and received signal, to minimize the number of conductors required on the instrument portion 30. These can be connected or integrated into the CMUTs in a flip-chip bonding approach, or located adjacent and connected using more traditional interconnections (e.g., wire bonding, or solder connections to a multi-layer printed circuit board). In at least one embodiment, as illustrated in FIG. 23, the distal end 34 of the instrument portion 30 provides a plurality of arrays 50 of transducers 36 with each array 50 separately multiplexed, which allows treatment of a large tissue range (e.g., large tumor) even without motion, and without the wiring burden associated with traditional single-wire-per-transducer 36 configurations. Additionally, in at least one embodiment, as illustrated in FIG. 24, each transducer 36 provides a plurality of independent transducer cells 120 where regions carry out different functions. In at least one such embodiment, some of the transducer cells 120 (for example, the transducer cells 120 depicted as filled circles in FIG. 24) are configured for obtaining the at least one ultrasound image of the target material 26 (along with surrounding tissue 40), while other of the transducer cells 120 (for example, the transducer cells 120 depicted as empty circles in FIG. 24) are configured for selectively emitting acoustic energy to heat, destroy and/or perturb the target material 26. In at least one such embodiment, the transducer cells 120 operate simultaneously or intermittently relative to each other.

In several embodiments described above, use of non-acoustic sensors are used to enhance the apparatus 20. In at least one such embodiment, where each transducer 36 is a CMUT, these sensors, including temperature and force sensors, can be integrated into the same fabrication as the CMUT. In several embodiments described above, particularly where the instrument portion 30 is configured as a needle, needle flexibility is required. In at least one such embodiment, the transducers 36 are mounted or otherwise connected to an at least one flexible conductive backing 88 (similar to flexible PCBs), thereby allowing the backing 88 (along with the transducers 36) to be wrapped around the instrument portion 30, as illustrated in FIG. 25. In further embodiments, each of the at least one backing 88 may take on any other sizes, shapes, dimensions, configurations, relative positions, and/or quantities, now known or later developed, so long as the apparatus 20 is capable of substantially carrying out the functionality described herein. In at least one such embodiment, known methods and techniques for creating flexible transducers 36 may be utilized, while in further embodiments, any other method or technique later developed may also be utilized. In at least one alternate embodiment, as illustrated in FIG. 26, the instrument portion 30 provides a plurality of substantially laterally-oriented, spaced apart grooves 90 positioned and configured for allowing a length of the instrument portion 30 to resiliently flex along the grooves 90. In at least one such embodiment, the transducers 36 are positioned along the distal end 34 of the instrument portion 30 between one or more of the spaced apart grooves 90 (i.e., the transducers 36 are positioned on the non-flexing portions of the instrument portion 30). In further embodiments, the grooves 90 may take on any other sizes, shapes, dimensions, configurations, relative positions, and/or quantities, now known or later developed, so long as the apparatus 20 is capable of substantially carrying out the functionality described herein.

As mentioned above, in at least one embodiment, the at least one transducer 36 is configured for operating in a pulse-echo configuration, which enables the apparatus 20 to capture acoustic information from the surrounding tissue 40 near the distal end 34 of the instrument portion 30. The high-frequency content of this data may then be used to discriminate the different tissue 40 and the target material 26, thus providing the information needed to navigate the instrument portion 30 to the desired final position in relation to the target material 26. A number of processing techniques of this pulse-echo data (including but not limited to quantitative ultrasound, harmonic imaging, and statistical analysis) as well as modes of operation (including but not limited to doppler, plane wave imaging, shear wave imaging) can improve the discrimination ability of the apparatus 20 based on the at least one ultrasound image captured by the at least one transducer 36. A key advantage of such embodiments is that, because the distal end 34 of the instrument portion 30 is near the tissue 40 and target material 26, both traditional diagnostic frequencies (5-20 MHz) as well as very high frequency ultrasound can be used (20-70 MHz) to resolve finer detail of the surrounding tissue 40, which would not be accessible to prior art external ultrasound equipment due to the poor penetration depth of high frequency. Further, because a range of frequencies are available for analysis, use of frequency dependent tissue 40 properties can allow for increased discrimination ability.

Additionally, because certain target material 26 (such as cancerous lesions, for example) are often characterized by an increase in local stiffness, a number of mechanical features can be incorporated into the apparatus 20 to support stiffness estimation. In at least one such embodiment, the instrument portion 30 incorporates an at least one active mechanical deformation element (such as an embedded force sensor 92, for example), which can apply forces to the local tissue 40. Using the at least one transducer 36 to image the tissue 40 throughout this applied deformation, along with image processing techniques to align the image regions over the course of the deformation, can enable estimation of the spatial distribution of tissue 40 stiffness throughout the local region of tissue 40. Embedded force sensors 92 can also improve the stiffness estimate by providing known forces at the distal end 34 of the instrument portion 30. Again, this stiffness estimation map can be combined with other imaging results and presented to a user of the apparatus 20 to allow an assessment of the location of the instrument portion 30 relative to the target material 26 and surrounding anatomy.

The position and orientation estimate of the instrument portion 30 may also be enhanced through several methods; namely, the registration of a preoperative image and/or one or more intraoperative imaging modalities (such as x-ray, intraoperative CT, traditional 2d/3d ultrasound, video and stereo imaging, and depth imaging, for example). These additional imaging modalities, which provide additional information as to the location of anatomic configuration of the target material 26 within the body, can be aligned using several methods based on image content, or user-aided steps such as the manual identification of anatomic landmarks. The localization of the at least one ultrasound image captured by the at least one transducer 36 with the additional set of imaging can be supported by dedicated real-time tool tracking hardware, such as magnetic sensors 114 (FIG. 19) integrated into the apparatus 20, or optically-localized tracking markers (such as stereo tracking markers) attached to the sidewall 46 of the instrument portion 30). The combination of these imaging sources can provide additional estimates as to the initial entry point into the tissue 40 to minimize collateral tissue 40 damage, as well as localization with respect to anatomy and pathology. They can also assist in understanding the curvature, and thus the applied force against the tissue 40. Various methods can also be used to continually improve the registration due to the introduced tissue 40 deformation or fracture caused by the instrument portion 30, including deformable tissue 40 models supplemented by estimated tissue 40 stiffnesses provided by the various imaging modalities including on-tip imaging (i.e., the at least one ultrasound image obtained by the at least one transducer 36 positioned on the distal end 34 of the instrument portion 30). A key advantage of such embodiments is that the tip-based ultrasound provides continuous direct, high quality imaging information relating to the clinical task (positioning the distal end 34 of the instrument portion 30 with respect to anatomy) which then reduces the localization burden on other imaging modalities, and does not require use of assumptions with known inaccuracies (e.g., no deformations of the apparatus 20, or no deformations or changes in tissue 40).

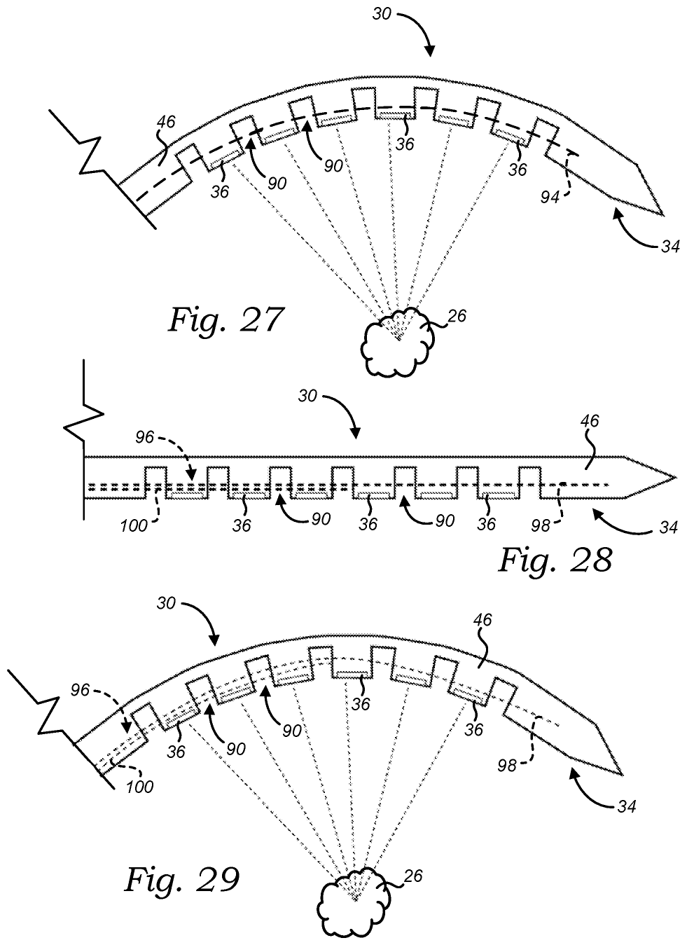

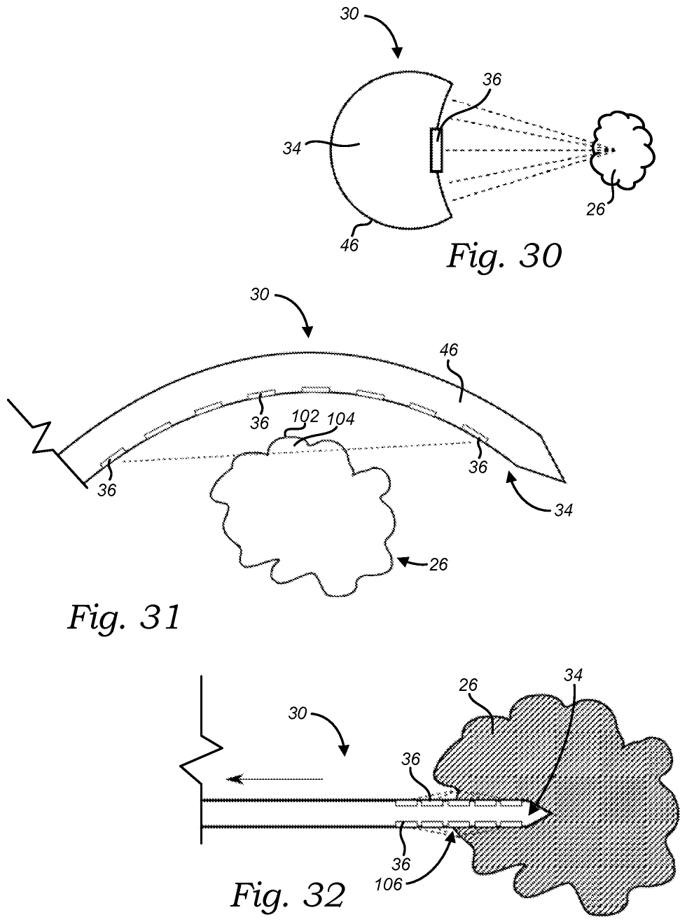

In at least one embodiment, based on the local tissue 40 data provided by the at least one transducer 36, the apparatus 20 precisely focuses acoustic energy to achieve the desired ablation shape. Focusing and steering energy at the distal end 34 of the instrument portion 30 is important to achieve a precise ablation region. Known prior art devices and techniques primarily depend on conductive thermal propagation to achieve the desired size of the ablation. However, as tissue 40 coagulation occurs, thermal propagation is significantly reduced, requiring the use of very high temperatures (often greater than 80-90 degrees Celsius) to achieve the needed ablation sizes. These high temperatures further limit the ability to precisely deliver ablation, and increase the risk of using such devices near critical anatomy. In at least one embodiment, the apparatus 20 is capable of precisely focusing acoustic energy using one or more of phase modulation, frequency modulation, and pulse shape modulation. Modulating these parameters allows the shaping of an ablation zone in multiple degrees of freedom (e.g., penetration depth, axial and lateral ablation width). In at least one further embodiment, in addition to focusing acoustic energy using one or more of the above noted electrical means, focusing of acoustic energy may be achieved via combined mechanical means such as articulation to change focal depth and rotation of sensors. For example, in at least one embodiment, the distal end 34 of the instrument portion 30 is flexible and configured for selectively moving between a substantially planar shape (FIG. 26) and a substantially hemispherical shape (FIG. 27), thereby leading to various focus depths. In at least one such embodiment, as illustrated in FIGS. 26 and 27, the distal end 34 of the instrument portion 30 provides an at least one internal wire 94 (such as nitinol wire, for example) positioned and configured for selectively articulating the distal end 34 of the instrument portion 30 (where the at least one transducer 36 is positioned) into a hemispherical shape, where the focus depth is now dependent on the strain on the wire 94. In further embodiments, the at least one wire 94 may take on any other sizes, shapes, dimensions, configurations, relative positions, and/or quantities, now known or later developed, so long as the apparatus 20 is capable of substantially carrying out the functionality described herein. In at least one alternate embodiment, as illustrated in FIGS. 28 and 29, the distal end 34 of the instrument portion 30 provides an at least one pull cable 96 positioned and configured for selectively articulating the distal end 34 of the instrument portion 30 into a hemispherical shape. In at least one such embodiment, the distal end 34 provides a first pull cable 98 and a second pull cable 100 attached at different points within the distal end 34 so as to increase a number of points of articulation in the distal end 34, thereby allowing the apparatus 20 to selectively apply different forces to the tissue 40. In further embodiments, the at least one pull cable 96 may take on any other sizes, shapes, dimensions, configurations, relative positions, and/or quantities, now known or later developed, so long as the apparatus 20 is capable of substantially carrying out the functionality described herein. In at least one further alternate embodiment, the distal end 34 of the instrument portion 30 is pre-stressed so as to achieve a substantially hemispherical shape after extending through a curved path in tissue 40. In still further embodiments, any other mechanism—now known or later developed—capable of selectively moving the distal end 34 between a relatively planar shape and a relatively hemispherical shape may be substituted. In at least one embodiment, manipulation of the distal end 34 of the instrument portion 30 is achieved manually, while in at least one alternate embodiment, the distal end 34 is manipulated automatically, such as by a robotic system for example. The focusing techniques utilized by the apparatus 20 are dependent at least in part of the type and location of the target material 26, along with the configuration of the apparatus 20. For example, where the transducers 36 are arranged in a “forward facing” configuration, focusing will be primarily electronic in at least one embodiment. As another example, where the distal end 34 of the instrument portion 30 is configured for curving around the target material 26, electronic and mechanical focusing can be combined to provide adequate coverage of the target material 26. In at least one alternate embodiment, as illustrated in length and a circumference 0, the distal end 34 of the instrument portion 30 is rigidly configured as a substantially hemispherical shape in cross-section. In at least one embodiment, as illustrated in FIG. 31, the hemispherical shape of the distal end 34 is such that the instrument portion 30 is capable of achieving transmissive propagation of the ultrasound. Such embodiments allow for yet another method to control the ablated target material 26, by first focusing the ultrasound energy to ablate a desired external boundary 102 of the target material 26, and then ablating an enclosed area 104 of the target material 26. This is a more precise method to deliver therapy as ablated tissue 40 has reduced thermal propagation properties; thus ablating outside-in (versus the traditional inside-out method) provides additional confidence as the ablated boundary would naturally reduce the thermal propagation outside of the target material 26.

As discussed above, in at least one embodiment, the at least one first transducer 42 is configured for obtaining an at least one ultrasound image of the target material 26 (along with surrounding tissue 40), while the at least one second transducer 44 is configured for selectively emitting acoustic energy to heat, destroy and/or perturb the target material 26. In at least one such embodiment, the at least one first transducer 42 operates simultaneously or intermittently relative to the operation of the at least one second transducer 44. Additionally, in at least one such embodiment, the apparatus 20 is capable of identifying the heated zones by sensing the changes in acoustic properties via a pulse-echo technique. The speed of sound and acoustic attenuation of tissue 40 is temperature dependent, thus impacting the reflected (“echo”) signal as the temperature changes. In addition, as tissue 40 coagulates its mechanical properties change. Thus as part of the emitted pulse in at least one embodiment, shear waves are induced in the tissue 40 of interest (i.e., the tissue 40 proximal to the target material 26) to measure the elasticity or stiffness of the tissue 40. In another embodiment, the distal end 34 of the instrument portion 30 is configured for selectively applying a mechanical stress in the region of interest to cause small deformations that can then be processed to monitor the stiffness change due to the ablation. This deformation portion may be the same deformation actuation that enables mechanical focusing. These applied mechanical stresses may also be monitored by a collection of embedded force sensors 92, as well as displacement sensors on the actuation method, to provide the means to estimate stiffness. Because many of the acoustic properties relate to an assumption of the speed of sound through distinct tissue 40 types, an initial ultrasound image, along with an estimation of tissue 40 types being imaged, may improve the estimation of the speed of sound, and thus the positional accuracy of the ultrasound image. In at least one embodiment, as energy is delivered and tissue 40 properties change, the initial ultrasound image can be registered (i.e., aligned) with subsequent ultrasound images, with a deformable image model. The degree of deformation needed relates to both the speed of sound changes as well as geometric changes in the tissue 40 (e.g., tissue 40 expansion). Use of the deformation signal can thus provide additional information as to geometric changes if speed of sound changes due to temperature are derived from another source or estimated thermal model. In at least one embodiment, using additional ultrasound modes (such as quantitative ultrasound, or doppler, for example) to further improve the spatial estimation of tissue 40 types (and thus the speed of sound) can further enhance the ultrasound image. Further, in at least one embodiment, a mechanical deformation or reconfiguration of the instrument portion 30 such that some of the transducers 36 lie in a straight line configuration (i.e., not pulse-echo, but transmissive) to other transducers 36 can enable direct estimation of speed of sound through tissue 40. The method by which this mechanical deformation occurs can also be the actuation in certain embodiments as described herein for navigation to region of interest, stiffness estimation, mechanical focusing, or shear wave generation. Embodiments where the at least one transducer 36 is a CMUT in close proximity to the tissue 40 of interest have the unique advantage of being able to capture wideband data from a more homogeneous region when compared to traditional ultrasound thermometry techniques. Reflected wideband acoustic data that has not propagated through multiple tissue 40 types is key to accurate local ablation monitoring as many temperature effects are tissue 40 dependent, non-linear, and frequency dependent. For example, many tissues 40 exhibit a shift in reflected acoustic energy between speckle generators (i.e., sub-wavelength components in tissue 40 that are highly reflective) and normal tissue 40, depending on temperature and frequency. By examining these distributions over time, in at least one embodiment, the apparatus 20 can estimate the temperature throughout the tissue 40.

To further improve the accuracy of the ablation monitoring, in at least one embodiment, a thermal propagation model is used to process the acoustic data—either locally by the apparatus 20 or by the at least one computing device 22 in selective communication with the apparatus 20. An initial ultrasound image that combines quantitative ultrasound with doppler ultrasound techniques will provide an estimate of the thermal properties of the region of interest by identifying the various tissue 40 types. This information combined with the sensing of the changing acoustic properties increases the signal to noise ratio by taking into account the relative change in temperature specifically due to the rate of delivered energy. This model may be continuously updated with deformation estimates from registration, estimates from intensity analysis over time, mechanical deformation aided tissue 40 property estimates, and direct measurements of temperature using a collection of embedded temperature sensors. This method thereby significantly improves the accuracy of the ablation zone monitoring. Accuracy at temperatures between 40-55 degrees Celsius is important for the precision delivery of ablation therapy. While cell death starts to occur at ˜43 degrees Celsius, phase changes in tissue 40 occur past that point (˜60 degrees Celsius), changing tissue 40 thermal propagation properties. For many tissues 40, this change limits the amount of thermal energy propagated, which can increase the burden on the apparatus 20. All of the described methods above can also be used to estimate tissue 40 phase change (for further confirmation of ablation zone coverage and cell death). Because the apparatus 20 has the ability to deliver pressures that can cause mechanical changes in the tissue 40 (e.g., cavitation) in at least one embodiment, this can additionally be used to enhance the ability to interrogate temperature changes, where the apparatus 20 generates cavitation bubbles with one acoustic intensity, then monitor the resorption rate (using imaging) which will correlate with temperature or tissue 40 function or water content.

In at least one embodiment, such real-time ablation monitoring can be used to directly guide the ablation, as opposed to simply presenting the information to the user (i.e., surgeon, clinician, etc.). This, combined with tissue 40 type discrimination, means that the desired area will be ablated precisely, completely destroying the target material 26 and preserving nearby healthy tissue 40. In at least one such embodiment, an additional optimal control component is provided by the apparatus 20, where the optimal heat application from the instrument portion 30 is calculated to bring about the desired ablation zone. Multiple approaches can be utilized in such embodiments, including but not limited to optimal thermal control strategies, treating the region as a lumped thermal model, and running multiple versions of a forward-running simulation and choosing the one with the most ideal outcome, and/or choosing the one that matches closest with observation. In such embodiments, the user is able to input a desired ablation plan, and maintain supervisory control during the execution of that plan. Because the apparatus 20 has the ability to deliver pressures that can cause mechanical changes in the tissue 40 (e.g., cavitation) in at least one embodiment, this can additionally be used to enhance the ability to deliver energy more efficiently into tissue 40, where the apparatus 20 generates cavitation bubbles initially, then those bubbles absorb and heat incoming ultrasound pressure waves more efficiently than tissue 40 alone.

In at least one embodiment, the precision ablation capabilities of the apparatus 20 may be utilized in the context of nerve ablation, where neural structures are destroyed to change regulation of a particular body function. While the embodiments described herein can be used to target neural anatomy as well as pathology, the ultrasound functionality of the instrument portion 30 can also be used to deliver energy to stimulate nerves. This stimulation can be used to localize nerves of a particular function, or assess nerve function. This assessment can help guide and inform a user during an ablation procedure.

Additionally, in at least one embodiment, as illustrated in FIG. 32, where the instrument portion 30 is configured as a needle (so as to create an entry wound 100 in the tissue 40 upon being inserted therewithin), the acoustic energy emitted by the at least one transducer 36 to heat, destroy and/or perturb the target material 26 is also capable of sealing the entry wound 106 upon the distal end 34 of the instrument portion 30 subsequently being withdrawn from the tissue 40. In a bit more detail, as the distal end 34 of the instrument portion 30 is withdrawn from the tissue 40, the at least one transducer 36 is capable of delivering a sufficient amount of acoustic energy to coagulate the blood proximal to the entry wound 106.

Aspects of the present specification may also be described as the following embodiments:

1. An ultrasound ablation apparatus for facilitating navigation, ablation, and ablation monitoring relative to an at least one target material positioned within or adjacent to a tissue of a patient, the apparatus comprising: an instrument portion; a base portion engaged with a proximal end of the instrument portion; an opposing distal end of the instrument portion providing a plurality of ultrasound transducers configured for both obtaining an at least one ultrasound image of the target material and selectively emitting acoustic energy to heat, destroy and/or perturb the target material, the transducers arranged so as to form an at least one array; and the distal end of the instrument portion further providing an at least one acoustically matched covering positioned and configured for extending over top of the transducers so as to not inhibit the functionality of the transducers; whereby, the apparatus is capable of precisely focusing acoustic energy toward the target material to achieve a desired ablation shape, based on data gathered from the at least one ultrasound image.

2. The ultrasound ablation apparatus according to embodiment 1, wherein the base portion is removably engaged with the proximal end of the instrument portion.

3. The ultrasound ablation apparatus according to embodiments 1-2, wherein the base portion is permanently engaged or otherwise integral with the proximal end of the instrument portion.

4. The ultrasound ablation apparatus according to embodiments 1-3, wherein the transducers are configured for operating in a pulse-echo configuration.

5. The ultrasound ablation apparatus according to embodiments 1-4, wherein the plurality of transducers includes an at least one first transducer configured for obtaining the at least one ultrasound image of the target material, and an at least one second transducer configured for selectively emitting acoustic energy to heat, destroy and/or perturb the target material.

6. The ultrasound ablation apparatus according to embodiments 1-5, wherein the at least one first transducer and at least one second transducer are arranged in an alternating pattern on the distal end of the instrument portion.

7. The ultrasound ablation apparatus according to embodiments 1-6, wherein each of the transducers is a high-bandwidth, low-profile, high-efficiency transducer.

8. The ultrasound ablation apparatus according to embodiments 1-7, wherein each of the transducers is a capacitive micromachined ultrasound transducer (“CMUT”).

9. The ultrasound ablation apparatus according to embodiments 1-8, wherein the at least one array is positioned on a sidewall of the instrument portion so as to be arranged in a substantially “side facing” configuration.

10. The ultrasound ablation apparatus according to embodiments 1-9, wherein the at least one array is positioned on a terminal face of the distal end of the instrument portion so as to be arranged in a substantially “forward facing” configuration.

11. The ultrasound ablation apparatus according to embodiments 1-10, wherein the transducers are arranged in a one-dimensional array along a length of the sidewall.

12. The ultrasound ablation apparatus according to embodiments 1-11, wherein the transducers are arranged in a two-dimensional array along both a length and a circumference of the sidewall.

13. The ultrasound ablation apparatus according to embodiments 1-12, wherein the sidewall provides an at least one substantially flat cut-out on which the at least one array of transducers is positioned.

14. The ultrasound ablation apparatus according to embodiments 1-13, wherein the at least one covering extends over top of the at least one cut-out and is shaped for retaining a substantially circular cross-section for the distal end of the instrument portion.

15. The ultrasound ablation apparatus according to embodiments 1-14, wherein the at least one covering is shaped as a substantially hemispherical dome and positioned to extend over top of the terminal face.

16. The ultrasound ablation apparatus according to embodiments 1-15, wherein the distal end of the instrument portion is positioned within an internal passage of a needle.

17. The ultrasound ablation apparatus according to embodiments 1-16, wherein the distal end of the instrument portion is configured for selectively extending a distance toward a terminal opening of the internal passage of the needle in order to observe, heat, destroy and/or perturb the target material when the needle is inserted into the tissue, then subsequently retracting back into the internal passage prior to the needle being withdrawn from the tissue.

18. The ultrasound ablation apparatus according to embodiments 1-17, wherein the terminal face of the distal end of the instrument portion is configured as a stepped needle providing a plurality of circumferential, coaxially aligned steps oriented in a “forward facing” direction, with each of the steps having a diameter relatively smaller than a diameter of an immediately preceding one of the steps.

19. The ultrasound ablation apparatus according to embodiments 1-18, wherein the terminal face of the distal end of the instrument portion further provides a plurality of transducers radially arranged along each of the steps.

20. The ultrasound ablation apparatus according to embodiments 1-19, wherein the at least one covering is positioned and configured for extending over top of each of the steps and associated transducers so as to cooperate with the distal end of the instrument portion to form a substantially conical shape.

21. The ultrasound ablation apparatus according to embodiments 1-20, wherein the at least one covering is a rigid shield configured for being selectively retractable when the transducers are positioned proximal the target material, thereby temporarily exposing the transducers to a volume of bodily fluids surrounding the target material, such that the bodily fluids facilitate acoustic communication between the transducers and the target material.

22. The ultrasound ablation apparatus according to embodiments 1-21, wherein the distal end of the instrument portion is configured for selectively delivering an acoustic medium configured for facilitating acoustic communication between the transducers and the target material.

23. The ultrasound ablation apparatus according to embodiments 1-22, wherein the acoustic medium is at least one of a fluid and a gel.

24. The ultrasound ablation apparatus according to embodiments 1-23, wherein the acoustic medium is contained in an at least one reservoir in fluid communication with the distal end of the instrument portion via an at least one delivery channel extending therebetween.

25. The ultrasound ablation apparatus according to embodiments 1-24, wherein the sidewall provides a plurality of coverings each positioned and configured for extending over top of a subset of the transducers, with each of the coverings being in fluid communication with the at least one reservoir via a separate at least one delivery channel.

26. The ultrasound ablation apparatus according to embodiments 1-25, wherein at least one reservoir is positioned within at least one of the instrument portion and the base portion.

27. The ultrasound ablation apparatus according to embodiments 1-26, wherein the at least one reservoir is positioned external to the apparatus.

28. The ultrasound ablation apparatus according to embodiments 1-27, wherein the instrument portion provides a first delivery channel and a second delivery channel each extending between the at least one reservoir and the distal end of the instrument portion so as to circulate the acoustic medium therethrough.

29. The ultrasound ablation apparatus according to embodiments 1-28, wherein the distal end of the instrument portion further provides an at least one temperature sensor positioned and configured for detecting a temperature of the distal end of the instrument portion along with the tissue proximal thereto, whereby, upon the detected temperature reaching a pre-defined threshold, the apparatus is configured for selectively circulating the acoustic medium through the distal end of the instrument portion so as to cool the tissue proximal to the distal end of the instrument portion.

30. The ultrasound ablation apparatus according to embodiments 1-29, wherein the at least one temperature sensor is positioned within the at least one covering.

31. The ultrasound ablation apparatus according to embodiments 1-30, wherein each of the first delivery channel and second delivery channel terminates proximal one of a first end or an opposing second end of the array of transducers.

32. The ultrasound ablation apparatus according to embodiments 1-31, wherein: the first delivery channel terminates proximal a first end of the array of transducers; and the second delivery channel terminates proximal an opposing second end of the array of transducers.

33. The ultrasound ablation apparatus according to embodiments 1-32, wherein the at least one covering is a deformable enclosure configured for maintaining the acoustic medium over top of the transducers.

34. The ultrasound ablation apparatus according to embodiments 1-33, wherein the enclosure is in fluid communication with the at least one delivery channel.

35. The ultrasound ablation apparatus according to embodiments 1-34, wherein the enclosure is pre-filled with the acoustic medium.

36. The ultrasound ablation apparatus according to embodiments 1-35, wherein the enclosure is constructed out of a solid, deformable acoustic medium.

37. The ultrasound ablation apparatus according to embodiments 1-36, wherein the distal end of the instrument portion further provides an at least one contact sensor positioned and configured for detecting the presence of acoustic contact between the transducers and the target material.

38. The ultrasound ablation apparatus according to embodiments 1-37, wherein the at least one contact sensor is positioned within the at least one covering.

39. The ultrasound ablation apparatus according to embodiments 1-38, wherein the instrument portion further provides an at least one magnetic sensor.

40. The ultrasound ablation apparatus according to embodiments 1-39, wherein the instrument portion is configured as a needle.

41. The ultrasound ablation apparatus according to embodiments 1-40, wherein the instrument portion is configured for being inserted into and traversing through a catheter, endoscope or similar structure.

42. The ultrasound ablation apparatus according to embodiments 1-41, wherein the instrument portion is configured as a laparoscopic surgical tool.

43. The ultrasound ablation apparatus according to embodiments 1-42, wherein the base portion provides an insertion motor in mechanical communication with the instrument portion and configured for selectively driving the distal end of the instrument portion a distance into, and subsequently out of, the tissue during use of the apparatus.

44. The ultrasound ablation apparatus according to embodiments 1-43, wherein the base portion provides a rotation motor in mechanical communication with the instrument portion and configured for selectively rotating the distal end of the instrument portion in order to align the at least one transducer with the target material.

45. The ultrasound ablation apparatus according to embodiments 1-44, wherein the instrument portion provides a plurality of conductors positioned and configured for electrically interconnecting an at least one electronic driver with the transducers.

46. The ultrasound ablation apparatus according to embodiments 1-45, wherein the transducers are multiplexed, such that a first set of wires allows access to each of an at least one row of the at least one array of transducers, and a second set of wires allows access to each of an at least one column of the at least one array of transducers.

47. The ultrasound ablation apparatus according to embodiments 1-46, wherein the distal end of the instrument portion provides a plurality of arrays of transducers with each array separately multiplexed.

48. The ultrasound ablation apparatus according to embodiments 1-47, wherein each transducer provides a plurality of independent transducer cells.

49. The ultrasound ablation apparatus according to embodiments 1-48, wherein some of the transducer cells are configured for obtaining the at least one ultrasound image of the target material, while other of the transducer cells are configured for selectively emitting acoustic energy to heat, destroy and/or perturb the target material.

50. The ultrasound ablation apparatus according to embodiments 1-49, wherein the transducers are mounted or otherwise connected to a flexible conductive backing, thereby allowing the backing, along with the transducers, to be wrapped circumferentially around the distal end of the instrument portion.

51. The ultrasound ablation apparatus according to embodiments 1-50, wherein the distal end of the instrument portion is flexible and configured for selectively moving between a substantially planar shape and a substantially hemispherical shape.

52. The ultrasound ablation apparatus according to embodiments 1-51, wherein the instrument portion provides a plurality of substantially laterally-oriented, spaced apart grooves positioned and configured for allowing a length of the instrument portion to resiliently flex along the grooves.

53. The ultrasound ablation apparatus according to embodiments 1-52, wherein the transducers are positioned along the distal end of the instrument portion between one or more of the spaced apart grooves.

54. The ultrasound ablation apparatus according to embodiments 1-53, wherein the distal end of the instrument portion provides an internal wire positioned and configured for selectively articulating the distal end of the instrument portion into the hemispherical shape.

55. The ultrasound ablation apparatus according to embodiments 1-54, wherein the distal end of the instrument portion provides an at least one pull cable positioned and configured for selectively articulating the distal end of the instrument portion into the hemispherical shape.

56. The ultrasound ablation apparatus according to embodiments 1-55, wherein the distal end of the instrument portion provides a first pull cable and a second pull cable attached at different points within the distal end, thereby increasing a number of points of articulation in the distal end.

57. The ultrasound ablation apparatus according to embodiments 1-56, wherein the distal end of the instrument portion is pre-stressed so as to achieve a substantially hemispherical shape after extending through a curved path in the tissue.

58. The ultrasound ablation apparatus according to embodiments 1-57, wherein the instrument portion provides an at least one active mechanical deformation element positioned and configured for selectively applying forces to the tissue proximal the target material.

59. The ultrasound ablation apparatus according to embodiments 1-58, wherein the at least one active mechanical deformation element is a force sensor embedded within the distal end of the instrument portion.

60. The ultrasound ablation apparatus according to embodiments 1-59, wherein the distal end of the instrument portion is rigidly configured as a substantially hemispherical shape in cross-section so as to achieve transmissive propagation of the ultrasound.

61. The ultrasound ablation apparatus according to embodiments 1-60, wherein the apparatus is in selective communication with each of a computing device and an imaging display.

62. An ultrasound ablation apparatus for facilitating navigation, ablation, and ablation monitoring relative to an at least one target material positioned within or adjacent to a tissue of a patient, the apparatus comprising: an instrument portion; a base portion engaged with a proximal end of the instrument portion; an opposing distal end of the instrument portion providing a plurality of ultrasound transducers configured for both obtaining an at least one ultrasound image of the target material and selectively emitting acoustic energy to heat and destroy the target material, the transducers arranged so as to form an at least one array positioned on a sidewall of the instrument portion; the sidewall providing an at least one acoustically matched covering positioned and configured for extending over top of the transducers so as to not inhibit the functionality of the transducers while retaining a substantially circular cross-section for the distal end of the instrument portion; and the at least one covering in fluid communication with an at least one reservoir containing an acoustic medium configured for facilitating acoustic communication between the transducers and the target material; whereby, the apparatus is capable of precisely focusing acoustic energy toward the target material to achieve a desired ablation shape, based on data gathered from the at least one ultrasound image.

63. An ultrasound ablation apparatus for facilitating navigation, ablation, and ablation monitoring relative to an at least one target material positioned within or adjacent to a tissue of a patient, the apparatus comprising: an instrument portion; a base portion engaged with a proximal end of the instrument portion; an opposing distal end of the instrument portion providing a plurality of ultrasound transducers configured for both obtaining an at least one ultrasound image of the target material and selectively emitting acoustic energy to heat and destroy the target material, the transducers arranged so as to form an at least one array positioned on a terminal face of the distal end of the instrument portion; the terminal face of the distal end of the instrument portion providing an at least one acoustically matched covering positioned and configured for extending over top of the transducers so as to not inhibit the functionality of the transducers; and the at least one covering in fluid communication with an at least one reservoir containing an acoustic medium configured for facilitating acoustic communication between the transducers and the target material; whereby, the apparatus is capable of precisely focusing acoustic energy toward the target material to achieve a desired ablation shape, based on data gathered from the at least one ultrasound image.

64. An ultrasound ablation system for facilitating navigation, ablation, and ablation monitoring relative to an at least one target material positioned within or adjacent to a tissue of a patient, the system comprising: an ultrasound ablation apparatus comprising: an instrument portion; a base portion engaged with a proximal end of the instrument portion; an opposing distal end of the instrument portion providing a plurality of ultrasound transducers configured for both obtaining an at least one ultrasound image of the target material and selectively emitting acoustic energy to heat, destroy and/or perturb the target material, the transducers arranged so as to form an at least one array; and the distal end of the instrument portion further providing an at least one acoustically matched covering positioned and configured for extending over top of the transducers so as to not inhibit the functionality of the transducers; and an imaging display in selective communication with the ultrasound ablation apparatus and configured for receiving and displaying the ultrasound images transmitted by the ultrasound ablation apparatus; whereby, the ultrasound ablation apparatus is capable of precisely focusing acoustic energy toward the target material to achieve a desired ablation shape, based on data gathered from the at least one ultrasound image.

65. The ultrasound ablation system according to embodiment 64, wherein the base portion is removably engaged with the proximal end of the instrument portion.

66. The ultrasound ablation system according to embodiments 64-65, wherein the base portion is permanently engaged or otherwise integral with the proximal end of the instrument portion.

67. The ultrasound ablation system according to embodiments 64-66, wherein the transducers are configured for operating in a pulse-echo configuration.

68. The ultrasound ablation system according to embodiments 64-67, wherein the plurality of transducers includes an at least one first transducer configured for obtaining the at least one ultrasound image of the target material, and an at least one second transducer configured for selectively emitting acoustic energy to heat, destroy and/or perturb the target material.

69. The ultrasound ablation system according to embodiments 64-68, wherein the at least one first transducer and at least one second transducer are arranged in an alternating pattern on the distal end of the instrument portion.

70. The ultrasound ablation system according to embodiments 64-69, wherein each of the transducers is a high-bandwidth, low-profile, high-efficiency transducer.

71. The ultrasound ablation system according to embodiments 64-70, wherein each of the transducers is a capacitive micromachined ultrasound transducer (“CMUT”).

72. The ultrasound ablation system according to embodiments 64-71, wherein the at least one array is positioned on a sidewall of the instrument portion so as to be arranged in a substantially “side facing” configuration.

73. The ultrasound ablation system according to embodiments 64-72, wherein the at least one array is positioned on a terminal face of the distal end of the instrument portion so as to be arranged in a substantially “forward facing” configuration.

74. The ultrasound ablation system according to embodiments 64-73, wherein the transducers are arranged in a one-dimensional array along a length of the sidewall.

75. The ultrasound ablation system according to embodiments 64-74, wherein the transducers are arranged in a two-dimensional array along both a length and a circumference of the sidewall.

76. The ultrasound ablation system according to embodiments 64-75, wherein the sidewall provides an at least one substantially flat cut-out on which the at least one array of transducers is positioned.

77. The ultrasound ablation system according to embodiments 64-76, wherein the at least one covering extends over top of the at least one cut-out and is shaped for retaining a substantially circular cross-section for the distal end of the instrument portion.

78. The ultrasound ablation system according to embodiments 64-77, wherein the at least one covering is shaped as a substantially hemispherical dome and positioned to extend over top of the terminal face.

79. The ultrasound ablation system according to embodiments 64-78, wherein the distal end of the instrument portion is positioned within an internal passage of a needle so as to be configured as a forward facing spherical probe.

80. The ultrasound ablation system according to embodiments 64-79, wherein the distal end of the instrument portion is configured for selectively extending a distance toward a terminal opening of the internal passage of the needle in order to observe, heat, destroy and/or perturb the target material when the needle is inserted into the tissue, then subsequently retracting back into the internal passage prior to the needle being withdrawn from the tissue.

81. The ultrasound ablation system according to embodiments 64-80, wherein the terminal face of the distal end of the instrument portion is configured as a stepped needle providing a plurality of circumferential, coaxially aligned steps oriented in a “forward facing” direction, with each of the steps having a diameter relatively smaller than a diameter of an immediately preceding one of the steps.

82. The ultrasound ablation system according to embodiments 64-81, wherein the terminal face of the distal end of the instrument portion further provides a plurality of transducers radially arranged along each of the steps.

83. The ultrasound ablation system according to embodiments 64-82, wherein the at least one covering is positioned and configured for extending over top of each of the steps and associated transducers so as to cooperate with the distal end of the instrument portion to form a substantially conical shape.

84. The ultrasound ablation system according to embodiments 64-83, wherein the at least one covering is a rigid shield configured for being selectively retractable when the transducers are positioned proximal the target material, thereby temporarily exposing the transducers to a volume of bodily fluids surrounding the target material, such that the bodily fluids facilitate acoustic communication between the transducers and the target material.

85. The ultrasound ablation system according to embodiments 64-84, wherein the distal end of the instrument portion is configured for selectively delivering an acoustic medium configured for facilitating acoustic communication between the transducers and the target material.

86. The ultrasound ablation system according to embodiments 64-85, wherein the acoustic medium is at least one of a fluid and a gel.

87. The ultrasound ablation system according to embodiments 64-86, wherein the acoustic medium is contained in an at least one reservoir in fluid communication with the distal end of the instrument portion via an at least one delivery channel extending therebetween.

88. The ultrasound ablation system according to embodiments 64-87, wherein the sidewall provides a plurality of coverings each positioned and configured for extending over top of a subset of the transducers, with each of the coverings being in fluid communication with the at least one reservoir via a separate at least one delivery channel.

89. The ultrasound ablation system according to embodiments 64-88, wherein at least one reservoir is positioned within at least one of the instrument portion and the base portion.

90. The ultrasound ablation system according to embodiments 64-89, wherein the at least one reservoir is positioned external to the apparatus.

91. The ultrasound ablation system according to embodiments 64-90, wherein the instrument portion provides a first delivery channel and a second delivery channel each extending between the at least one reservoir and the distal end of the instrument portion so as to circulate the acoustic medium therethrough.

92. The ultrasound ablation system according to embodiments 64-91, wherein the distal end of the instrument portion further provides an at least one temperature sensor positioned and configured for detecting a temperature of the distal end of the instrument portion along with the tissue proximal thereto, whereby, upon the detected temperature reaching a pre-defined threshold, the apparatus is configured for selectively circulating the acoustic medium through the distal end of the instrument portion so as to cool the tissue proximal to the distal end of the instrument portion.

93. The ultrasound ablation system according to embodiments 64-92, wherein the at least one temperature sensor is positioned within the at least one covering.

94. The ultrasound ablation system according to embodiments 64-93, wherein each of the first delivery channel and second delivery channel terminates proximal one of a first end or an opposing second end of the array of transducers.

95. The ultrasound ablation system according to embodiments 64-94, wherein: the first delivery channel terminates proximal a first end of the array of transducers; and the second delivery channel terminates proximal an opposing second end of the array of transducers.

96. The ultrasound ablation system according to embodiments 64-95, wherein the at least one covering is a deformable enclosure configured for maintaining the acoustic medium over top of the transducers.

97. The ultrasound ablation system according to embodiments 64-96, wherein the enclosure is in fluid communication with the at least one delivery channel.

98. The ultrasound ablation system according to embodiments 64-97, wherein the enclosure is pre-filled with the acoustic medium.

99. The ultrasound ablation system according to embodiments 64-98, wherein the enclosure is constructed out of a solid, deformable acoustic medium.

100. The ultrasound ablation system according to embodiments 64-99, wherein the distal end of the instrument portion further provides an at least one contact sensor positioned and configured for detecting the presence of acoustic contact between the transducers and the target material.

101. The ultrasound ablation system according to embodiments 64-100, wherein the at least one contact sensor is positioned within the at least one covering.

102. The ultrasound ablation system according to embodiments 64-101, wherein the instrument portion further provides an at least one magnetic sensor.

103. The ultrasound ablation system according to embodiments 64-102, wherein the instrument portion is configured as a needle.

104. The ultrasound ablation system according to embodiments 64-103, wherein the instrument portion is configured for being inserted into and traversing through a catheter, endoscope or similar structure.

105. The ultrasound ablation system according to embodiments 64-104, wherein the instrument portion is configured as a laparoscopic surgical tool.

106. The ultrasound ablation system according to embodiments 64-105, wherein the base portion provides an insertion motor in mechanical communication with the instrument portion and configured for selectively driving the distal end of the instrument portion a distance into, and subsequently out of, the tissue during use of the apparatus.

107. The ultrasound ablation system according to embodiments 64-106, wherein the base portion provides a rotation motor in mechanical communication with the instrument portion and configured for selectively rotating the distal end of the instrument portion in order to align the at least one transducer with the target material.

108. The ultrasound ablation system according to embodiments 64-107, wherein the instrument portion provides a plurality of conductors positioned and configured for electrically interconnecting an at least one electronic driver with the transducers.

109. The ultrasound ablation system according to embodiments 64-108, wherein the transducers are multiplexed, such that a first set of wires allows access to each of an at least one row of the at least one array of transducers, and a second set of wires allows access to each of an at least one column of the at least one array of transducers.

110. The ultrasound ablation system according to embodiments 64-109, wherein the distal end of the instrument portion provides a plurality of arrays of transducers with each array separately multiplexed.

111. The ultrasound ablation system according to embodiments 64-110, wherein each transducer provides a plurality of independent transducer cells.

112. The ultrasound ablation system according to embodiments 64-111, wherein some of the transducer cells are configured for obtaining the at least one ultrasound image of the target material, while other of the transducer cells are configured for selectively emitting acoustic energy to heat, destroy and/or perturb the target material.

113. The ultrasound ablation system according to embodiments 64-112, wherein the transducers are mounted or otherwise connected to a flexible conductive backing, thereby allowing the backing, along with the transducers, to be wrapped circumferentially around the distal end of the instrument portion.

114. The ultrasound ablation system according to embodiments 64-113, wherein the distal end of the instrument portion is flexible and configured for selectively moving between a substantially planar shape and a substantially hemispherical shape.

115. The ultrasound ablation system according to embodiments 64-114, wherein the instrument portion provides a plurality of substantially laterally-oriented, spaced apart grooves positioned and configured for allowing a length of the instrument portion to resiliently flex along the grooves.

116. The ultrasound ablation system according to embodiments 64-115, wherein the transducers are positioned along the distal end of the instrument portion between one or more of the spaced apart grooves.

117. The ultrasound ablation system according to embodiments 64-116, wherein the distal end of the instrument portion provides an internal wire positioned and configured for selectively articulating the distal end of the instrument portion into the hemispherical shape.

118. The ultrasound ablation system according to embodiments 64-117, wherein the distal end of the instrument portion provides an at least one pull cable positioned and configured for selectively articulating the distal end of the instrument portion into the hemispherical shape.

119. The ultrasound ablation system according to embodiments 64-118, wherein the distal end of the instrument portion provides a first pull cable and a second pull cable attached at different points within the distal end, thereby increasing a number of points of articulation in the distal end.

120. The ultrasound ablation system according to embodiments 64-119, wherein the distal end of the instrument portion is pre-stressed so as to achieve a substantially hemispherical shape after extending through a curved path in the tissue.

121. The ultrasound ablation system according to embodiments 64-120, wherein the instrument portion provides an at least one active mechanical deformation element positioned and configured for selectively applying forces to the tissue proximal the target material.

122. The ultrasound ablation system according to embodiments 64-121, wherein the at least one active mechanical deformation element is a force sensor embedded within the distal end of the instrument portion.

123. The ultrasound ablation system according to embodiments 64-122, wherein the distal end of the instrument portion is rigidly configured as a substantially hemispherical shape in cross-section so as to achieve transmissive propagation of the ultrasound.

124. The ultrasound ablation system according to embodiments 64-123, further comprising a computing device in selective communication with at least one of the ultrasound ablation apparatus and the imaging display.

In closing, regarding the exemplary embodiments of the present invention as shown and described herein, it will be appreciated that an ultrasound ablation apparatus and associated methods of use are disclosed and configured for facilitating navigation, ablation, and ablation monitoring. Because the principles of the invention may be practiced in a number of configurations beyond those shown and described, it is to be understood that the invention is not in any way limited by the exemplary embodiments, but is generally directed to an ultrasound ablation apparatus and is able to take numerous forms to do so without departing from the spirit and scope of the invention. It will also be appreciated by those skilled in the art that the present invention is not limited to the particular geometries and materials of construction disclosed, but may instead entail other functionally comparable structures or materials, now known or later developed, without departing from the spirit and scope of the invention.

Certain embodiments of the present invention are described herein, including the best mode known to the inventor(s) for carrying out the invention. Of course, variations on these described embodiments will become apparent to those of ordinary skill in the art upon reading the foregoing description. The inventor(s) expect skilled artisans to employ such variations as appropriate, and the inventor(s) intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein. Similarly, as used herein, unless indicated to the contrary, the term “substantially” is a term of degree intended to indicate an approximation of the characteristic, item, quantity, parameter, property, or term so qualified, encompassing a range that can be understood and construed by those of ordinary skill in the art.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

The terms “a,” “an,” “the” and similar references used in the context of describing the present invention (especially in the context of the following claims) are to be construed to cover both the singular and the plural, unless otherwise indicated herein or clearly contradicted by context. Further, ordinal indicators—such as “first,” “second,” “third,” etc. —for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

When used in the claims, whether as filed or added per amendment, the open-ended transitional term “comprising” (along with equivalent open-ended transitional phrases thereof such as “including,” “containing” and “having”) encompasses all the expressly recited elements, limitations, steps and/or features alone or in combination with un-recited subject matter; the named elements, limitations and/or features are essential, but other unnamed elements, limitations and/or features may be added and still form a construct within the scope of the claim. Specific embodiments disclosed herein may be further limited in the claims using the closed-ended transitional phrases “consisting of” or “consisting essentially of” in lieu of or as an amendment for “comprising.” When used in the claims, whether as filed or added per amendment, the closed-ended transitional phrase “consisting of” excludes any element, limitation, step, or feature not expressly recited in the claims. The closed-ended transitional phrase “consisting essentially of” limits the scope of a claim to the expressly recited elements, limitations, steps and/or features and any other elements, limitations, steps and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps and/or features specifically recited in the claim, whereas the meaning of the closed-ended transitional phrase “consisting essentially of” is being defined as only including those elements, limitations, steps and/or features specifically recited in the claim and those elements, limitations, steps and/or features that do not materially affect the basic and novel characteristic(s) of the claimed subject matter. Therefore, the open-ended transitional phrase “comprising” (along with equivalent open-ended transitional phrases thereof) includes within its meaning, as a limiting case, claimed subject matter specified by the closed-ended transitional phrases “consisting of” or “consisting essentially of.” As such, embodiments described herein or so claimed with the phrase “comprising” are expressly or inherently unambiguously described, enabled and supported herein for the phrases “consisting essentially of” and “consisting of.”

Any claims intended to be treated under 35 U.S.C. § 112(f) will begin with the words “means for,” but use of the term “for” in any other context is not intended to invoke treatment under 35 U.S.C. § 112(f). Accordingly, Applicant reserves the right to pursue additional claims after filing this application, in either this application or in a continuing application.

It should be understood that the logic code, programs, modules, processes, methods, and the order in which the respective elements of each method are performed are purely exemplary. Depending on the implementation, they may be performed in any order or in parallel, unless indicated otherwise in the present disclosure. Further, the logic code is not related, or limited to any particular programming language, and may comprise one or more modules that execute on one or more processors in a distributed, non-distributed, or multiprocessing environment. Additionally, the various illustrative logical blocks, modules, methods, and algorithm processes and sequences described in connection with the embodiments disclosed herein can be implemented as electronic hardware, computer software, or combinations of both. To clearly illustrate this interchangeability of hardware and software, various illustrative components, blocks, modules, and process actions have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of this document.

The phrase “non-transitory,” in addition to having its ordinary meaning, as used in this document means “enduring or long-lived.” The phrase “non-transitory computer readable medium,” in addition to having its ordinary meaning, includes any and all computer readable mediums, with the sole exception of a transitory, propagating signal. This includes, by way of example and not limitation, non-transitory computer-readable mediums such as register memory, processor cache and random-access memory (“RAM”).

The methods as described above may be used in the fabrication of integrated circuit chips. The resulting integrated circuit chips can be distributed by the fabricator in raw wafer form (that is, as a single wafer that has multiple unpackaged chips), as a bare die, or in a packaged form. In the latter case, the chip is mounted in a single chip package (such as a plastic carrier, with leads that are affixed to a motherboard or other higher level carrier) or in a multi-chip package (such as a ceramic carrier that has either or both surface interconnections or buried interconnections). In any case, the chip is then integrated with other chips, discrete circuit elements, and/or other signal processing devices as part of either (a) an intermediate product, such as a motherboard, or (b) an end product. The end product can be any product that includes integrated circuit chips, ranging from toys and other low-end applications to advanced computer products having a display, a keyboard or other input device, and a central processor.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicant and does not constitute any admission as to the correctness of the dates or contents of these documents.

While aspects of the invention have been described with reference to at least one exemplary embodiment, it is to be clearly understood by those skilled in the art that the invention is not limited thereto. Rather, the scope of the invention is to be interpreted only in conjunction with the appended claims and it is made clear, here, that the inventor(s) believe that the claimed subject matter is the invention.

1. (canceled)

2. An ultrasound ablation and imaging system comprising:
an ablation unit comprising;
an instrument body configured to be inserted into a tissue, the instrument body comprising a proximal portion and a distal portion;
one or more ultrasound transducers coupled to the distal portion of the instrument body, wherein the one or more ultrasound transducers are configured for selectively emitting energy for obtaining one or more ultrasound images of surrounding tissue and/or heating, perturbing, or destroying a target tissue, the one or more transducer arranged to form one or more transducer arrays; and
a monitoring unit comprising;
one or more sensors, coupled to the distal portion, configured to measure one or more characteristics of the tissue, comprising, target and/or surrounding tissue, wherein the one or more sensors comprise at least one of the one or more ultrasound transducers; and
a processor configured to perform ablation monitoring, wherein the processor is configured to receive and process sensor data of the target tissue and/or surrounding tissue to provide real-time ablation monitoring.

3. The system of claim 2, wherein the one or more sensors comprise one or more temperature sensors, pressure sensors, force sensors, magnetic sensors, acoustic sensors, optical sensors, or displacement sensors.

4. The system of claim 2, wherein the processor is configured to determine a tissue property, the tissue property comprising one or more of an acoustic property, thermal property, thermal propagation property, or a mechanical property.

5. The system of claim 4, wherein the processor is configured to determine a change in the tissue property from the one or more measured characteristic.

6. The system of claim 2, wherein the processor is configured to receive an initial set of acoustic information and/or ultrasound image, and wherein the processor is further configured to use the measured one or more characteristics of the tissue to update a tissue model based on the initial set of set of acoustic information and/or ultrasound image.

7. The system of claim 6, wherein the tissue model comprises at least one of a thermal model, a thermal propagation model, or a deformable image model.

8. The system of claim 6, wherein the processor is configured to continuously update the tissue model.

9. The system in claim 6, wherein the processor is configured to use a thermal propagation model to process acoustic data and update a deformable image model to align subsequent ultrasound images with the initial ultrasound image.

10. The system of claim 9, wherein the processor is configured to update the thermal propagation model using one or more of a deformation estimate, an intensity analysis over time, or a temperature measurement.

11. The system of claim 2, wherein the processor is configured to determine one or more optimal thermal control strategies and control one or more parameters of the at least one or more ultrasound transducers to adjust an ablation zone.

12. The system of claim 2, wherein the processor is configured to adjust one or more acoustic energy parameters of or drive signals to the one or more transducers in response to a sensed signal.

13. The system of claim 2, wherein the processor is configured to one or more of (i) run a simulated ablation treatment to determine a particular ablation treatment having a desired outcome or (ii) receive a user input for a desired ablation treatment.

14. The system of claim 2, wherein the one or more ultrasound transducers are CMUTs.

15. The system of claim 2, wherein the one or more ultrasound transducers are configured to one or more of (i) perform ultrasound imaging (ii) measure one or more tissue properties or (iii) acoustically ablate tissue.

16. The system of claim 2, wherein the one or more ultrasound transducers are arranged in one or more arrays, each of the arrays comprising a first set of the ultrasound transducers configured to perform imaging and a second set of the ultrasound transducers configured to perform ablation.

17. The system of claim 2, wherein the one or more ultrasound transducers comprise a plurality of ultrasound transducers, wherein the plurality of ultrasound transducers are configured to be independently controlled to alternate between ultrasound imaging and acoustic ablation.

18. The system of claim 2, wherein the instrument body further comprises one or more segmented expandable coverings coupled to the distal portion of the instrument body, the expandable coverings extending over and encapsulating at least one of the one or more transducer arrays.

19. The system of claim 18, wherein the expandable coverings are disposed on one or more lateral sides of the instrument body.

20. The system of claim 18, wherein the expandable coverings extend circumferentially around the instrument body.

21. The system of claim 18, wherein the expandable coverings are segmented one or more of longitudinally or circumferentially.

22. The system of claim 21, wherein segments of the expandable coverings are configured to be selectively expanded, and wherein the segments are expanded by an acoustic medium stored in the fluid reservoir.

23. The system of claim 18, wherein the one or more expandable cover are coupled to one or more sensors, and wherein the expandable covers are configured to measure one or more of geometric data or stiffness data of the tissue.

24. The system of claim 18, wherein the expandable covers are configured to be inflated or deflated to adjust one or more ultrasound and/or acoustic signal parameters of the one or more transducers.

25. The system of claim 18, wherein the expandable covers are configured to be in inflated such that the temperature of the device and/or target tissue is reduced.

26. The system of claim 18, wherein the expandable covers are configured to change from a first position, wherein the expandable cover are flat against the distal portion, to a second position, wherein the expandable covers expand radially from the distal portion.
